(19) United States
(12) Patent Application Publication
(10) Pub. No.: US 2009/0286813 A1
(43) Pub. Date: Nov. 19, 2009

(54) Thioxanthine Derivatives and Their Use as Inhibitors of MPO

(75) Inventors: Donald Pivonka, Wilmington, DE (US); Anna-Karin Tiden, Sodertalje (SE); Jenny Viklund, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/296,347

(22) PCT Filed: Apr. 12, 2007
(86) PCT No.: PCT/SE2007/000348

§ 371 Date: Oct. 7, 2008

Related U.S. Application Data
(60) Provisional application No. 60/791,773, filed on Apr. 13, 2006.

(51) Int. Cl. A61K 31/52; C07D 473/00; A61P 29/00; A61P 9/00; A61P 11/00

(52) U.S. Cl. 514/263.2; 514/263.1; 544/264

(57) ABSTRACT

There are disclosed novel compounds of Formula (I) wherein R¹, X and Y are as defined in the specification, and pharmaceutically acceptable salts thereof; together with processes for their preparation, compositions containing them and their use in therapy. The compounds are inhibitors of the enzyme MPO and are thereby particularly useful in the treatment or prophylaxis of neuroinflammatory disorders, cardio- and cerebrovascular atherosclerotic disorders and peripheral artery disease and respiratory disorders.

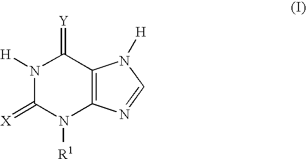

### FIELD OF THE INVENTION

The present invention relates to novel thioxanthine derivatives, processes for their preparation, compositions containing them and their use in therapy.

### BACKGROUND OF THE INVENTION

Myeloperoxidase (MPO) is a heme-containing enzyme found predominantly in polymorphonuclear leukocytes (PMNs). MPO is one member of a diverse protein family of mammalian peroxidases that also includes eosinophil peroxidase, thyroid peroxidase, salivary peroxidase, lactoperoxidase, prostaglandin H synthase, and others. The mature enzyme is a dimer of identical halves. Each half molecule contains a covalently bound heme that exhibits unusual spectral properties responsible for the characteristic green colour of MPO. Cleavage of the disulphide bridge linking the two halves of MPO yields the hemi-enzyme that exhibits spectral and catalytic properties indistinguishable from those of the intact enzyme. The enzyme uses hydrogen peroxide to oxidize chloride to hypochlorous acid. Other halides and pseudohalides (like thiocyanate) are also physiological substrates to MPO.

PMNs are of particular importance for combating infections. These cells contain MPO, with well-documented microbicidal action. PMNs act non-specifically by phagocytosis to engulf microorganisms, incorporate them into vacuoles, termed phagosomes, which fuse with granules containing myeloperoxidase to form phagolysosomes. In phagolysosomes the enzymatic activity of the myeloperoxidase leads to the formation of hypochlorous acid, a potent bactericidal compound. Hypochlorous acid is oxidizing in itself, and reacts most avidly with thiols and thioethers, but also converts amines into chloramines, and chlorinates aromatic amino acids. Macrophages are large phagocytic cells, which, like PMNs, are capable of phagocytosing microorganisms. Macrophages can generate hydrogen peroxide and upon activation also produce myeloperoxidase. MPO and hydrogen peroxide can also be released to the outside of the cells where the reaction with chloride can induce damage to adjacent tissue.

Linkage of myeloperoxidase activity to disease has been implicated in neurological diseases with a neuroinflammatory response including multiple sclerosis, Alzheimer's disease, Parkinson's disease and stroke as well as other inflammatory diseases or conditions like asthma, chronic obstructive pulmonary disease, cystic fibrosis, atherosclerosis, ischemic heart disease, heart failure, inflammatory bowel disease, renal glomerular damage and rheumatoid arthritis. Lung cancer has also been suggested to be associated with high MPO levels.

### Multiple Sclerosis (MS)

MPO positive cells are immensely present in the circulation and in tissue undergoing inflammation. More specifically MPO containing macrophages and microglia has been documented in the CNS during disease; multiple sclerosis (Nagra R M, et al. Journal of Neuroimmunology 1997; 78(1-2): 97-107), Parkinson's disease (Choi D-K. et al. J. Neurosci. 2005; 25(28): 6594-600) and Alzheimer's disease (Green P S. et al. Journal of Neurochemistry. 2004; 90(3): 724-33). It is supposed that some aspects of a chronic ongoing inflammation result in an overwhelming destruction where agents from MPO reactions have an important role.

The enzyme is released both extracellularly as well as into phagolysosomes in the neutrophils (Hampton M B, Kettle A J, Winterbourn C C. Blood 1998; 92(9): 3007-17). A prerequisite for the MPO activity is the presence of hydrogen peroxide, generated by NADPH oxidase and a subsequent superoxide dismutation. The oxidized enzyme is capable to use a plethora of different substrates of which chloride is most recognized. From this reaction the strong non-radical oxidant—hypochlorous acid (HOCl)—is formed. HOCl oxidizes sulphur containing amino acids like cysteine and methionine very efficiently (Peskin A V, Winterbourn C C. Free Radical Biology and Medicine 2001; 30(5): 572-9). It also forms chloramines with amino groups, both in proteins and other biomolecules (Peskin A V. et al. Free Radical Biology and Medicine 2004; 37(10): 1622-30). It chlorinates phenols (like tyrosine) (Hazen S L. et al. Mass Free Radical Biology and Medicine 1997; 23(6): 909-16) and unsaturated bonds in lipids (Albert C J. et al. J. Biol. Chem. 2001; 276(26): 23733-41), oxidizes iron centers (Rosen H, Klebanoff S J. Journal of Biological Chemistry 1982; 257(22): 13731-354) and crosslinks proteins (Fu X, Mueller D M, Heinecke J W. Biochemistry 2002; 41(4): 1293-301).

Proteolytic cascades participate both in cell infiltration through the BBB as well as the destruction of BBB, myelin and nerve cells (Cuzner M L, Opdenakker G. Journal of Neuroimmunology 1999; 94(1-2): 1-14; Yong V W. et al. Nature Reviews Neuroscience 2001; 2(7): 502-11.). Activation of matrix metalloproteinases (MMPs) can be accomplished through the action of upstream proteases in a cascade as well as through oxidation of a disulfide bridge Fu X. et al. J. Biol. Chem. 2001; 276(44): 41279-87; Gu Z. et al. Science 2002; 297(5584): 1186-90). This oxidation can be either a nitrosylation or HOCl-mediated oxidation. Both reactions can be a consequence of MPO activity. Several reports have suggested a role for MMP's in general and MMP-9 in particular as influencing cell infiltration as well as tissue damage (BBB breakdown and deinyelination), both in MS and EAE (for review see Yong V W. et al, supra). The importance of these specific kinds of mechanisms in MS comes from studies where increased activity and presence of proteases have been identified in MS brain tissue and CSF. Supportive data has also been generated by doing EAE studies with mice deficient in some of the proteases implicated to participate in the MS pathology, or by using pharmacological approaches. The demyelination is supposed to be dependent on the cytotoxic T-cells and toxic products generated by activated phagocytes (Lassmann H. J Neurol Neurosurg Psychiatry 2003; 74(6): 695-7). The axonal loss is thus influenced by proteases and reactive oxygen and nitrogen intermediates. When MPO is present it will obviously have the capability of both activating proteases (directly as well as through disinhibition by influencing protease inhibitors) and generating reactive species.

### Chronic Obstructive Pulmonary Disease (COPD)

Chronic obstructive pulmonary disease (COPD) is a disease state characterised by airflow limitation that is not fully reversible. The airflow limitation is usually both progressive and associated with an abnormal inflammatory response of the lungs to noxious particles or gases. COPD is a major public health problem. It is the fourth leading cause of chronic morbidity and mortality in the United States and is projected to rank fifth in 2020 as a worldwide burden of disease. In the UK the prevalence of COPD is 1.7% in men and 1.4% in women. COPD spans a range of severity from mild to very severe, with the cost of treatment rising rapidly as the severity increases.

Levels of MPO in sputum and BAL are much greater in COPD patients that normal, non-smoking controls (Keatings V. M., Barnes P. J. Am. J Respir Crit. Care Med 1997; 155:449-453; Pesci, A. et al. Eur Respir J 1998; 12: 380-386). MPO levels are further elevated during exacerbations of the disease (Fiorini G. et al. Biomedicine & Pharmacotherapy 2000; 54:274-278; Crooks S. W. et al. European Respiratory Journal. 15(2):274-80, 2000). The role of MPO is likely to be more important in exacerbations of COPD (Sharon S. D. et al. Am J Respir Crit. Care Med. 2001; 163:349-355).

In addition to the destructive capacity of MPO there is a strong clinical link with vascular disease (Baldus S. et al. Circulation 2003; 108: 1440-5). Dysfunctional MPO polymorphisms are associated with a reduced risk of mortality from coronary artery disease (Nikpoor B. et al. Am Heart J 2001; 142: 336), and patients with high serum levels of MPO have increased risk of acute coronary syndrome. The effects of MPO on vascular disease may extend to COPD, since there is strong evidence that the pulmonary vasculature is one of the earliest sites of involvement in the smokers' lung. Striking changes in the intima of the pulmonary arteries have been described which show a dose relationship with smoking (Hale K. A., Niewoehner D. E., Cosio M. G. Am Rev Resp Dis 1980; 122: 273-8). The physiological function of MPO is associated with innate host defense. This role, however, is not critical as most cases of MPO deficient patients have relatively benign symptoms (Parry M. F. et al. Ann Int Med. 1981; 95: 293-301, Yang, K. D., Hill, H. R. Pediatr Infect Dis J. 2001; 20: 889-900). In summary, there is considerable evidence that elevated MPO levels in COPD may contribute to the disease via several mechanisms. A selective inhibitor of MPO would therefore be expected to alleviate both the acute and chronic inflammatory aspects of COPD and may reduce the development of emphysema.

### Atherosclerosis

An MPO inhibitor should reduce the atherosclerotic burden and/or the vulnerability of existing atherosclerotic lesions and thereby decrease the risk of acute myocardial infarction, unstable angina or stroke and reduce ischemia/reperfusion injury during acute coronary syndrome and ischemic cerebrovascular events. Several lines of data support a role for MPO in atherosclerosis. MPO is expressed in the shoulder regions and necrotic core of human atherosclerotic lesions and active enzyme has been isolated from autopsy specimens of human lesions (Daugherty, A. et al. (1994) J Clin Invest 94(1): 437-44). In eroded and ruptured human lesions, as compared to fatty streaks, an increased number of MPO expressing macrophages have been demonstrated, suggesting a particular role for MPO in acute coronary syndromes (Sugiyama, S. et al. (2001) Am J Pathol 158(3): 879-91). Patients with established coronary artery disease have higher plasma and leukocyte MPO levels than healthy controls (Zhang, R. et al. (2001) Jama 286(17): 2136-42). Moreover, in two large prospective studies plasma levels of MPO predicted the risk of future coronary events or revascularisation (Baldus, S. et al. (2003) Circulation 108(12): 1440-5; Brennan, M. et al. (2003) N Engl J Med 349(17): 1595-604). Total MPO deficiency in humans has a prevalence of 1 in 2000-4000 individuals. These individuals appear principally healthy but a few cases of severe Candida infection have been reported. Interestingly, MPO deficient humans are less affected by cardiovascular disease than controls with normal MPO levels (Kutter, D. et al. (2000) Acta Haematol 104(1)). A polymorphism in the MPO promoter affects expression leading to high and low MPO expressing individuals. In three different studies the high expression genotype has been associated with an increased risk of cardiovascular disease (Nikpoor, B. et al. (2001) Am Heart J 142(2): 336-9; Makela, R., P. J. Karhunen, et al. (2003) Lab Invest 83(7): 919-25; Asselbergs, F. W., et al. (2004) Am J Med 116(6): 429-30). Data accumulated during the last ten years indicate that the proatherogenic actions of MPO include oxidation of lipoproteins, induction of endothelial dysfunction via consuming nitric oxide and destabilisation of atherosclerotic lesions by activation of proteases (Nicholls, S. J. and S. L. Hazen (2005) Arterioscler Thromb Vasc Biol 25(6): 1102-11). Recently, several studies have focused on nitro- and chlorotyrosine modifications of LDL and HDL lipoproteins. Since chlorotyrosine modifications in vivo only can be generated by hypochlorus acid produced by MPO these modifications are regarded as specific markers of MPO activity (Hazen, S. L. and J. W. Heinecke (1997) J Clin Invest 99(9): 2075-81). LDL particles exposed to MPO in vitro become aggregated, leading to facilitated uptake via macrophage scavenger receptors and foam cell formation (Hazell, L. J. and R. Stocker (1993) Biochem J 290 (Pt 1): 165-72). Chlorotyrosine modification of apoA1, the main apolipoprotein of HDL cholesterol, results in impaired cholesterol acceptor function (Bergt, C., S. et al. (2004) Proc Natl Acad Sci USA; Zheng, L. et al. (2004) J Clin Invest 114(4): 529-41). Systematic studies of these mechanisms have shown that MPO binds to and travels with apoA1 in plasma. Moreover, MPO specifically targets those tyrosine residues of apoA1 that physically interact with the macrophage ABCA1 cassette transporter during cholesterol efflux from the macrophage (Bergt, C. et al. (2004) J Biol Chem 279(9): 7856-66; Shao, B. et al. (2005) J Biol Chem 280(7): 5983-93; Zheng et al. (2005) J Biol Chem 280(1): 38-47). Thus, MPO seems to have a dual aggravating role in atherosclerotic lesions, i.e. increasing lipid accumulation via aggregation of LDL particles and decreasing the reverse cholesterol transport via attack on the HDL protein apoA1.

The present invention discloses novel thioxanthines that surprisingly display useful properties as inhibitors of the enzyme MPO. Furthermore, the novel compounds of the present invention display either one or more than one of the following: (i) improved selectivity towards TPO; (ii) unexpectedly high inhibitory activity towards MPO; (iii) improved brain permeability; (iv) improved solubility and/or (v) improved half-life; when compared to known thioxanthines. Such thioxanthines are disclosed in e.g. WO 03/089430 and WO 05/037835.

### DISCLOSURE OF THE INVENTION

According to the present invention, there is provided a compound of Formula (I):

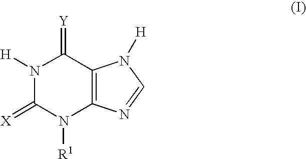

wherein
at least one of X and Y represents S, and the other represents O or S;
R¹ represents an aromatic ring system selected from phenyl, biphenyl, naphthyl or 5 or 6 membered heteroaromatic ring containing one or more heteroatoms selected from N, O or S and said 5 or 6 membered heteroaromatic ring may optionally be fused with a 5 or 6 membered saturated, partially saturated or unsaturated ring containing one or more atoms selected from C, N, O or S, and said ring system (said 5 or 6 membered heteroaromatic ring alone, or said 5 or 6 membered heteroaromatic ring fused with a 5 or 6 membered saturated, partially saturated or unsaturated ring) being optionally substituted by one or more substituents independently selected from halogen, CHF₂, CH₂F, CF₃, SO₍ₙ₎R², SO₍ₙ₎NR²R³, S(O)ₙ, OH, OCF₃, C1 to 6 alkyl, C1 to 6 alkoxy, CN, CONR⁴R⁵, NR⁴COR⁵ and COR⁵; said alkoxy being optionally further substituted by C1 to 6 alkoxy and said alkoxy optionally incorporating a carbonyl adjacent to the oxygen, and said alkyl being optionally further substituted by hydroxy or C1 to 6 alkoxy and said alkyl or alkoxy optionally incorporating a carbonyl adjacent to the oxygen or at any position in the alkyl; provided that when R¹ is phenyl, said phenyl must be substituted with one or more substituents independently selected from halogen, CHF₂, CH₂F, CF₃, SO₍ₙ₎R², SO₍ₙ₎NR²R³, OH, OCF₃, C1 to 6 alkyl, C1 to 6 alkoxy, CN, CONR⁴R⁵, NR⁴COR⁵ and COR⁵; said alkoxy being optionally further substituted by C1 to 6 alkoxy and said alkoxy optionally incorporating a carbonyl adjacent to the oxygen, and said alkyl being optionally further substituted by hydroxy or C1 to 6 alkoxy and said alkyl or alkoxy optionally incorporating a carbonyl adjacent to the oxygen or at any position in the alkyl;
at each occurrence, R², R³, R⁴ and R⁵ independently represent hydrogen, C1 to 6 alkyl or C1 to 6 alkoxy said alkoxy optionally incorporating a carbonyl adjacent to the oxygen, said alkyl being optionally further substituted by halogen, C1 to 6 alkoxy, CHO, C2 to 6 alkanoyl, OH, CONR⁶R⁷ and NR⁶COR⁷;
or the groups NR⁴R⁵ and NR²R³ each independently represent a 5 to 7 membered saturated azacyclic ring optionally incorporating one additional heteroatom selected from O, S and NR⁸, said ring being optionally further substituted by halogen, C1 to 6 alkoxy, CHO, C2 to 6 alkanoyl, OH, CONR⁶R⁷ and NR⁶COR⁷;
at each occurrence R⁶, R⁷ and R⁸ independently represent hydrogen or C1 to 6 alkyl, or the group NR⁶R⁷ represents a 5 to 7 membered saturated azacyclic ring optionally incorporating one additional heteroatom selected from O, S and NR⁸;
n is an integer 0, 1 or 2;
as a pharmaceutically acceptable salt, solvate or solvate of a salt thereof.

In one aspect of the invention, there is provided compounds of Formula (I), wherein X represents S and Y represents O.

In another aspect of the invention, there is provided compounds of Formula (I), wherein R¹ is a phenyl, substituted with one or more substituents independently selected from halogen, CHF₂, CH₂F, CF₃, SO₍ₙ₎R², SO₍ₙ₎NR²R³, OH, OCF₃, C1 to 6 alkyl, C1 to 6 alkoxy, CN, CONR⁴R⁵, NR⁴COR⁵ and COR⁵.

In yet another aspect of the invention, there is provided compounds of Formula (I), wherein R¹ is a phenyl substituted with one or two substituent selected from OCF₃, CN, halogen, methoxy and C1 to 6 alkyl.

In yet another aspect of the invention, there is provided compounds of Formula (I), wherein R¹ represents pyridyl optionally substituted by one or more substituents independently selected from halogen, CF₃, OCF₃, C1 to 6 alkyl and C1 to 6 alkoxy.

In yet another aspect of the invention, there is provided compounds of Formula (I), said compounds being:
3-(3-chlorophenyl)-2-thioxo-1,2,3,7-tetrahydro-6H-purin-6-one;
3-(3-ethylphenyl)-2-thioxo-1,2,3,7-tetrahydro-6H-purin-6-one;
2-thioxo-3-[3-(trifluoromethoxy)phenyl]-1,2,3,7-tetrahydro-6H-purin-6-one;
3-(4-chlorophenyl)-2-thioxo-1,2,3,7-tetrahydro-6H-purin-6-one;
3-(3,5-dichlorophenyl)-2-thioxo-1,2,3,7-tetrahydro-6H-purin-6-one;
3-(6-oxo-2-thioxo-1,2,6,7-tetrahydro-3H-purin-3-yl)benzonitrile;
3-(4-methoxyphenyl)-2-thioxo-1,2,3,7-tetrahydro-6H-purin-6-one;
3-quinolin-3-yl-2-thioxo-1,2,3,7-tetrahydro-6H-purin-6-one;
3-(2,5-difluorophenyl)-2-thioxo-1,2,3,7-tetrahydro-6H-purin-6-one;
3-(3-fluorophenyl)-2-thioxo-1,2,3,7-tetrahydro-6H-purin-6-one;
3-(2-chlorophenyl)-2-thioxo-1,2,3,7-tetrahydro-6H-purin-6-one;
3-(2-methoxyphenyl)-2-thioxo-1,2,3,7-tetrahydro-6H-purin-6-one;
2-thioxo-3-[6-(trifluoromethyl)pyridin-3-yl]-1,2,3,7-tetrahydro-6H-purin-6-one and
3-pyridin-3-yl-2-thioxo-1,2,3,7-tetrahydro-6H-purin-6-one;
   as a pharmaceutically acceptable salt, solvate or solvate of a salt thereof.

The compounds of Formula (I) may exist in enantiomeric forms. It is to be understood that all enantiomers, diastereomers, racemates, tautomers and mixtures thereof are included within the scope of the invention.

The compounds of Formula (I) may exist in tautomeric forms. All such tautomers and mixtures of tautomers are included within the scope of the present invention.

Unless otherwise indicated, the term “C1 to 6 alkyl” referred to herein denotes a straight or branched chain alkyl group having from 1 to 6 carbon atoms. Examples of such groups include methyl, ethyl, 1-propyl, n-butyl, iso-butyl, tert-butyl, pentyl and hexyl. The term “C1 to 7 alkyl” is to be interpreted analogously

Unless otherwise indicated, the term “C1 to 6 alkoxy” referred to herein denotes a straight or branched chain alkoxy group having from 1 to 6 carbon atoms. Examples of such groups include methoxy, ethoxy, 1-propoxy, 2-propoxy, tert-butoxy and pentoxy. The term “C1 to 7 alkoxy” is to be interpreted analogously.

Unless otherwise indicated, the term “C2 to 6 alkanoyl” referred to herein denotes a straight or branched chain alkyl group having from 1 to 5 carbon atoms with optional position on the alkyl group by a carbonyl group. Examples of such groups include acetyl, propionyl and pivaloyl.

Unless otherwise indicated, the term “halogen” referred to herein denotes fluoro, chloro, bromo and iodo.

Examples of a “5 or 6 membered heteroaromatic ring containing one or more heteroatoms selected from N, O or S” include, but is not limited to, pyrrole, oxazole, isoxazole, furazan, thiazole, imidazole, pyrazole, triazole, tetrazole, pyridine, pyrazine, pyrimidine and pyridazine.

Examples of a “5 or 6 membered saturated, partially saturated or unsaturated ring containing one or more atoms selected from C, N, O or S” include, but is not limited to, cyclopropyl, cyclopentyl, cyclohexyl, cyclohexenyl, cyclopentanone, tetrahydrofuran, pyrrolidine, piperidine, tetrahydropyridine, morpholine, piperazine, pyrrolidinone and piperidinone.

Examples of a “5 or 6 membered heteroaromatic ring containing one or more heteroatoms selected from N, O or S” when fused with a “5 or 6 membered saturated, partially saturated or unsaturated ring containing one or more atoms selected from C, N, O or S” include, but is not limited to, indole, isoindole and benzimidazole.

Examples of a 5 to 7 membered saturated azacyclic ring optionally incorporating one additional heteroatom selected from O, S and NR¹¹ include pyrrolidine, piperidine, piperazine, morpholine and thiomorpholine.

A further aspect of the invention is the use of the novel compounds of Formula (I) as a medicament.

A further aspect of the invention is the use of a compound of Formula (I), or a pharmaceutically acceptable salt thereof, in the manufacture of a medicament, for the treatment or prophylaxis of diseases or conditions in which inhibition of the enzyme MPO is beneficial.

A further aspect of the invention provides the use of a compound of Formula (I), or a pharmaceutically acceptable salt thereof, in the manufacture of a medicament, for the treatment or prophylaxis of neuroinflammatory disorders, cardio- and cerebrovascular atherosclerotic disorders and peripheral artery disease and respiratory disorders such as chronic obstructive pulmonary disease (COPD). According to the present invention, COPD is intended to include bronchitis, including infectious and eosinophilic bronchitis; emphysema; bronchiectasis or cystic fibrosis.

Another further aspect of the invention provides the use of a compound of Formula (I), or a pharmaceutically acceptable salt thereof, in the manufacture of a medicament, for the treatment or prophylaxis of multiple sclerosis. Treatment may include slowing progression of disease.

Another further aspect of the invention provides the use of a compound of Formula (I), or a pharmaceutically acceptable salt thereof, in the manufacture of a medicament, for the treatment or prophylaxis of Parkinson's disease. Treatment may include slowing progression of disease.

Another further aspect of the present invention provides the use of a compound of Formula (I) or a pharmaceutically acceptable salt thereof, in the manufacture of a medicament, for the treatment or prophylaxis of atherosclerosis by preventing and/or reducing the formation of new atherosclerotic lesions or plaques and/or by preventing or slowing progression of existing lesions and plaques.

Another further aspect of the present invention provides the use of a compound of Formula (I) or a pharmaceutically acceptable salt thereof, in the manufacture of a medicament, for the treatment or prophylaxis of atherosclerosis by changing the composition of the plaques to reduce the risk of plaque rupture and atherothrombotic events.

Another further aspect of the present invention provides the use of a compound of Formula (I) or a pharmaceutically acceptable salt thereof, in the manufacture of a medicament, for the treatment or prophylaxis of respiratory disorders, such as chronic obstructive pulmonary disease. Treatment may include slowing progression of disease.

According to the invention, there is also provided a method of treating, or reducing the risk of, diseases or conditions in which inhibition of the enzyme MPO is beneficial which comprises administering to a person suffering from or at risk of, said disease or condition, a therapeutically effective amount of a compound of Formula (I), or a pharmaceutically acceptable salt thereof.

Further, there is also provided a method of treating, or reducing the risk of, neuroinflammatory disorders, cardio- and cerebrovascular atherosclerotic disorders or peripheral artery disease, or heart failure, or respiratory disorders, such as chronic obstructive pulmonary disease (COPD), in a person suffering from or at risk of, said disease or condition, wherein the method comprises administering to the person a therapeutically effective amount of a compound of Formula (I), or a pharmaceutically acceptable salt thereof.

Further, there is also provided a method of treating, or reducing the risk of, multiple sclerosis in a person suffering from or at risk of, said disease or condition, wherein the method comprises administering to the person a therapeutically effective amount of a compound of Formula (I), or a pharmaceutically acceptable salt thereof.

Further, there is also provided a method of treating, or reducing the risk of, Parkinson's disease in a person suffering from or at risk of, said disease or condition, wherein the method comprises administering to the person a therapeutically effective amount of a compound of Formula (I), or a pharmaceutically acceptable salt thereof.

There is also provided a method of treating, or reducing the risk of atherosclerosis by preventing and/or reducing the formation of new atherosclerotic lesions or plaques and/or by preventing or slowing progression of existing lesions and plaques in a person suffering from or at risk of, said disease or condition, wherein the method comprises administering to the person a therapeutically effective amount of a compound of Formula (I) or a pharmaceutically acceptable salt thereof.

There is also provided a method of treating, or reducing the risk of atherosclerosis by changing the composition of the plaques so as to reduce the risk of plaque rupture and atherothrombotic events in a person suffering from or at risk of, said disease or condition, wherein the method comprises administering to the person a therapeutically effective amount of a compound of Formula (I) or a pharmaceutically acceptable salt thereof.

In another aspect the invention provides a pharmaceutical formulation comprising a therapeutically effective amount of a compound of Formula (I), or a pharmaceutically acceptable salt thereof, in admixture with a pharmaceutically acceptable adjuvant, diluent or carrier, for use in the treatment or prophylaxis of diseases or conditions in which inhibition of the enzyme MPO is beneficial.

In a further aspect the invention provides a pharmaceutical formulation comprising a therapeutically effective amount of a compound of Formula (I), or a pharmaceutically acceptable salt thereof, in admixture with a pharmaceutically acceptable adjuvant, diluent or carrier, for use in the treatment or prophylaxis of neuroinflammatory disorders.

In a further aspect the invention provides a pharmaceutical formulation comprising a therapeutically effective amount of a compound of Formula (I), or a pharmaceutically acceptable salt thereof, in admixture with a pharmaceutically acceptable adjuvant, diluent or carrier, for use in the treatment or prophylaxis of multiple sclerosis, cardio- and cerebrovascular atherosclerotic disorders and peripheral and heart failure disease and respiratory disorders, such as chronic obstructive pulmonary disease.

In another aspect the present invention provides a pharmaceutical formulation comprising a therapeutically effective amount of a compound of Formula (I), or a pharmaceutically acceptable salt thereof, in admixture with a pharmaceutically acceptable adjuvant, diluent or carrier, for use in the treatment or prophylaxis of atherosclerosis by preventing and reducing the formation of new atherosclerotic lesions and/or plaques and/or by preventing or slowing progression of existing lesions and plaques.

In another aspect the present invention provides a pharmaceutical formulation comprising a therapeutically effective amount of a compound of Formula (I), or a pharmaceutically acceptable salt thereof, in admixture with a pharmaceutically acceptable adjuvant, diluent or carrier, for use in the treatment or prophylaxis of atherosclerosis by changing the composition of the plaques so as to reduce the risk of plaque rupture and atherothrombotic events.

The present invention further relates to therapies for the treatment of: Neuroinflammatory Disorder(s) including but not limited to Multiple Sclerosis (MS), Parkinson's disease, Multiple System Atrophy (MSA), Corticobasal Degeneration, Progressive Supranuclear Paresis, Guillain-Barré Syndrome (GBS), chronic inflammatory demyelinating polyneuropathy (CIDP). Multiple sclerosis (MS) includes Relapse Remitting Multiple Sclerosis (RRMS), Secondary Progressive Multiple Sclerosis (SPMS), and Primary Progressive Multiple Sclerosis (PPMS).

The invention further relates to therapies for the treatment of:

Cognitive Disorder(s) including but not limited to
a) Dementia, including but not limited to Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Frontotemporal dementia Parkinson's Type (FTDP), progressive supranuclear palsy (PSP), Pick's Disease, Niemann-Pick's Disease, corticobasal degeneration, traumatic brain injury (TBI), dementia pugilistica, Creutzfeld-Jacob Disease and prion diseases;

### b) Cognitive Deficit in Schizophrenia (CDS);

### c) Mild Cognitive Impairment (MCI);

### d) Age-Associated Memory Impairment (AAMI);

### e) Age-Related Cognitive Decline (ARCD);

### f) Cognitive Impairment No Dementia (CIND).

The present invention further relates to therapies for the treatment of: Attention-Deficit and Disruptive Behavior Disorder(s) including but not limited to attention deficit disorder (ADD), attention deficit hyperactivity disorder (ADHD) and affective disorders.

The present invention also relates to the treatment of the diseases and conditions below which may be treated with the compounds of the present invention:

respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;

### Methods of Preparation

According to the invention, we further provide a process for the preparation of compounds of Formula (I), or a pharmaceutically acceptable salt, solvate, enantiomer, diastereomer or racemate thereof where R¹, X and Y are defined as in Formula (I).

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March 4th ed. McGraw Hill (1992) or, “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in Formula (I) except where defined differently. The terms “room temperature” and “ambient temperature” shall mean, unless otherwise specified, a temperature between 16 and 25° C. The term “reflux” shall mean, unless otherwise stated, in reference to an employed solvent using a temperature at or slightly above the boiling point of the named solvent. It is understood that microwaves can be used for the heating of reaction mixtures. The terms “flash chromatography” or “flash column chromatography” shall mean preparative chromatography on silica using an organic solvent, or mixtures thereof, as mobile phase.

### Preparation of End Products

1. A process for preparing a compound of Formula (I), wherein R¹ is defined as in Formula (I) and X is S and Y is O is shown in Scheme 1:

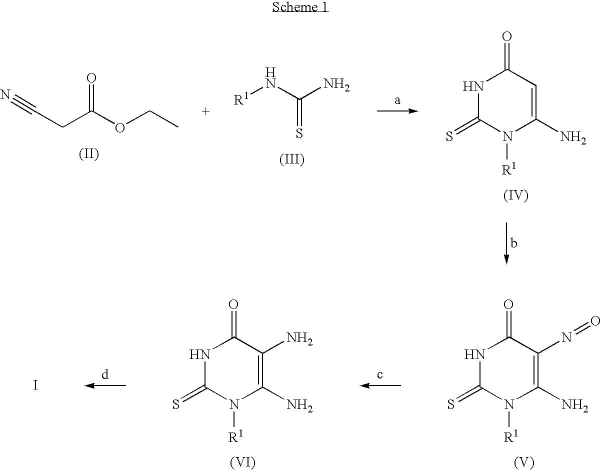

Compounds of formula (II), (III), (IV), (V) and (VI) are useful intermediates in the preparation of compound of Formula (I) wherein R¹ is defined as in Formula (I). Compounds of formula II-VI are either commercially available, or can be prepared from either commercially available, or in the literature described compounds. (Ouwerkerk et al. Eur. J. Org. Chem. 2002, 14, 2356).

a) Reaction of ethyl cyanoacetate (II) with a thiourea of formula (III) wherein R¹ is defined as in Formula (I). In the process, ethyl cyanoacetate (II) and an appropriate thiourea (III) are dissolved or suspended in a suitable alcohol such as ethanol and an alkoxide such as sodium ethoxide is added. The temperature is typically from 70° C. up to reflux temperature of the reaction mixture.

b) Reaction of a thiouracil of formula (IV), wherein R¹ is defined above with sodium nitrite in an acidic solution. In the process, the thiouracil (IV) is suspended in a solvent such as acetic acid (10 to 100%) or hydrochloric acid (aq. 1M) and stirred at a suitable temperature between 0° C. and 85° C. for 10 to 20 minutes before the sodium nitrite, dissolved in water, is added dropwise.

c) Reduction of a nitroso compound of formula (V), wherein R¹ is defined above. In the process, the reduction of the nitroso compound (V) may be carried out with a suitable reducing agent such as sodium dithionite in a suitable solvent mixture such as water and ammonia solution or sodium hydroxide (aq. 1N) at a temperature range between room temperature and 75° C. for 30 minutes to 24 hours. Alternatively the sodium dithionite could be added directly to the conditions used in step b.

d) The reaction of a diamine of formula (VI), wherein R¹ is defined above with i) formic acid, ii) formamidine acetate or with iii) trialkylorthoester is described below:

(i) In process (d), the diamine (VI) is treated with formic acid (98%), at a suitable temperature between ambient temperature and the reflux temperature of the reaction mixture. The process is continued for a suitable period of time, typically for between 20 to 30 minutes. After removal of the formic acid, treatment with a suitable aqueous base, for example, with 10% aqueous sodium hydroxide solution, then yields the compound of Formula (I). The treatment with base is carried out for a suitable time at a suitable temperature, for example for about 30 minutes to 90 minutes at a temperature between ambient temperature and the reflux temperature of the reaction mixture. Alternatively the reaction can be performed in a solvent such as water to which formic acid and sulphuric acid were added. The reaction was heated under reflux overnight which after neutralization gave the compound of Formula (I).
(ii) In process (d), the diamine (VI) is treated with formamidine acetate in a solvent such as DMSO at a suitable temperature, for example 70° C., until the reaction is complete, typically for 1-3 h.
(iii) In process (d), the diamine (VI) is treated at a suitable temperature with an excess of an appropriate ortho ester such as triethylorthoformate or tripropylorthoformate, optionally in the presence of a suitable solvent such as an alcohol, until reaction is complete. The temperature is typically up to the reflux temperature of the reaction mixture, and reaction times are generally from 30 minutes to overnight.

Other methods for the conversion of a diamine of formula (VI) into a compound of Formula (I) are described in the literature and will be readily known to the person skilled in the art.

2. A process for the preparation of compounds of Formula (I), wherein R¹ is defined as in Formula (I), X represents O or S and Y represents S:

i) Reaction of a compound of Formula (I), wherein R¹ is defined as in Formula (I) and X is S and Y is O (described in Scheme 1) with a sulphurising compound such as Lawesson's reagent or phosphorus pentasulphide in a suitable dry organic solvent such as benzene, pyridine, toluene, xylene, tetrahydrofuran, dichloromethane or dioxane at a temperature between 30° C. and the reflux temperature of the solvent gives a compound of Formula (I), wherein X and Y is S (Müller et al. J. Med. Chem. 2002, 45, 3440).
ii) Reaction of a compound of Formula (I), wherein R¹ is defined as in Formula (I) and X is O and Y is O, (The starting material can be prepared according to in Scheme 1 using an urea as a starting material instead of a thiourea in step (a), with a sulphurising compound such as Lawesson's reagent or phosphorus pentasulphide in a suitable dry organic solvent such as benzene, pyridine, toluene, xylene, tetrahydrofuran, dichloromethane or dioxane at a temperature between 30° C. and the reflux temperature of the solvent gives a compound of Formula (I), wherein X is O and Y is S (Müller et al. J. Med. Chem. 2002, 45, 3440; Merlos et al. Eur. J. Med. Chem. 1990, 25, 653).

The resultant compound of Formula (I), or another salt thereof, can where necessary be converted into a pharmaceutically acceptable salt thereof; or converting the resultant compound of Formula (I) into a further compound of Formula (I); and where desired converting the resultant compound of Formula (I) into an optical isomer thereof.

The present invention includes compounds of Formula (I), in the form of salts. Suitable salts include those formed with organic or inorganic acids or organic or inorganic bases. Such salts will normally be pharmaceutically acceptable although salts of non-pharmaceutically acceptable acids or bases may be of utility in the preparation and purification of the compound in question. Thus, acid addition salts include inter alia those formed from hydrochloric acid. Base addition salts include those in which the cation is inter alia sodium or potassium.

The compounds of the invention and intermediates thereto may be isolated from their reaction mixtures and, if necessary further purified, by using standard techniques.

The compounds of Formula (I) may exist in enantiomeric forms. Therefore, all enantiomers, diastereomers, racemates, tautomers and mixtures thereof are included within the scope of the invention. The various optical isomers may be isolated by separation of a racemic mixture of the compounds using conventional techniques, for example, fractional crystallisation, or HPLC. Alternatively, the various optical isomers may be prepared directly using optically is active starting materials.

Intermediate compounds may also exist in enantiomeric forms and may be used as purified enantiomers, diastereomers, racemates or mixtures.

Intermediate compounds may also exist in tautomeric forms and may be used as purified tautomers or mixtures.

The compounds of Formula (I) and their pharmaceutically acceptable salts are useful because they possess pharmacological activity as inhibitors of the enzyme MPO.

The compounds of Formula (I) and their pharmaceutically acceptable salts are indicated for use in the treatment or prophylaxis of diseases or conditions in which modulation of the activity of the enzyme myeloperoxidase (MPO) is desirable. In particular, linkage of MPO activity to disease has been implicated in neuroinflammatory diseases. Therefore the compounds of the present invention are particularly indicated for use in the treatment of neuroinflammatory conditions or disorders in mammals including man. The compounds are also indicated to be useful in the treatment of cardio- and cerebrovascular atherosclerotic disorders or peripheral artery disease or heart failure. The compounds are also indicated to be useful in the treatment of respiratory disorders, such as disorders of the respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus. Such conditions or disorders will be readily apparent to the man skilled in the art.

Conditions or disorders that may be specifically mentioned include multiple sclerosis, Alzheimer's disease, Parkinson's disease, amyotrophic lateral sclerosis and stroke, as well as other inflammatory diseases or conditions such as asthma, chronic obstructive pulmonary disease, cystic fibrosis, idiopathic pulmonary fibrosis, acute respiratory distress syndrome, sinusitis, rhinitis, psoriasis, dermatitis, uveitis, gingivitis, atherosclerosis, myocardial infarction, stroke, coronary heart disease, ischaemic heart disease, restenosis, inflammatory bowel disease, renal glomerular damage, liver fibrosis, sepsis, proctitis, rheumatoid arthritis, and inflammation associated with reperfusion injury, spinal cord injury and tissue damage/scarring/adhesion/rejection. Lung cancer has also been suggested to be associated with high MPO levels. The compounds are also expected to be useful in the treatment of pain.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above-mentioned therapeutic indications, the dosage administered will, of course, vary with the compound employed, the mode of administration and the treatment desired. However, in general, satisfactory results are obtained when the compounds are administered at a dosage of the solid form of between 1 mg and 2000 mg per day.

The compounds of Formula (I) and pharmaceutically acceptable derivatives thereof, may be used on their own, or in the form of appropriate pharmaceutical compositions in which the compound or derivative is in admixture with a pharmaceutically acceptable adjuvant, diluent or carrier. Thus, another aspect of the invention concerns a pharmaceutical composition comprising a novel compound of Formula (I) or a pharmaceutically acceptable salt thereof, in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988. The pharmaceutical composition preferably comprises less than 80% and more preferably less than 50% of a compound of formulae (I), or a pharmaceutically acceptable salt thereof.

There is also provided a process for the preparation of such a pharmaceutical composition that comprises mixing the ingredients.

The invention further relates to combination therapies wherein a compound of Formula (I) or a pharmaceutically acceptable salt thereof, or a pharmaceutical composition or formulation comprising a compound of Formula (I) is administered concurrently or sequentially with therapy and/or an agent for the treatment of any one of cardio- and cerebrovascular atherosclerotic disorders and peripheral artery disease and heart failure. In particular, a compound of Formula (I) or a pharmaceutically acceptable salt thereof may be administered in association with compounds from one or more of the following groups:

1) anti-inflammatory agents, for example
a) NSAIDs (e.g. acetylsalicylic acid, ibuprofen, naproxen, flurbiprofen, diclofenac, indometacin);
b) leukotriene synthesis inhibitors (5-LO inhibitors e.g. AZD4407, Zileuton, licofelone, CJ13610, CJ13454; FLAP inhibitors e.g. BAY-Y-1015, DG-031, MK591, MK886, A81834; LTA4 hydrolase inhibitors e.g. SC56938, SC57461A);
c) leukotriene receptor antagonists (e.g. CP195543, amelubant, LY293111, accolate, MK571);
   2) anti-hypertensive agents, for example
a) beta-blockers (e.g. metoprolol, atenolol, sotalol);
b) angiotensin converting enzyme inhibitors (e.g. captopril, ramipril, quinapril, enalapril);
c) calcium channel blockers (e.g. verapamil, diltiazem, felodipine, amlodipine);
d) angiotensin II receptor antagonists (e.g. irbesartan, candesartan, telemisartan, losartan);
   3) anti-coagulantia, for example
a) thrombin inhibitors (e.g. ximelagatran), heparines, factor Xa inhibitors;
b) platelet aggregation inhibitors (e.g. clopidrogrel, ticlopidine, prasugel, AZ4160);
   4) modulators of lipid metabolism, for example
a) insulin sensitizers such as PPAR agonists (e.g. pioglitazone, rosiglitazone, Galida, niuraglitazaar, gefemrozil, fenofibrate);
b) HMG-CoA reductase inhibitors, statins (e.g. simvastatin, pravastatin, atorvaststin, rosuvastatin, fluvastatin);
c) cholesterol absorption inhibitors (e.g. ezetimibe);
d) IBAT inhibitors (e.g. AZD-7806);
e) LXR agonists (e.g. GW-683965A, T-0901317);
f) FXR receptor modulators;
g) phospholipase inhibitors;
   5) anti-anginal agents, for example, nitrates and nitrites;
   6) modulators of oxidative stress, for example, anti-oxidants (probucol).

The invention is illustrated, but in no way limited, by the following examples:

### General Methods

All solvents used were analytical grade and commercially available anhydrous solvents were routinely used for reactions. Reactions were typically run under an inert atmosphere of nitrogen or argon.

¹H and ¹³C NMR spectra were recorded at 400 MHz for proton and 100 MHz for carbon-13 either on a Varian Unity+ 400 NMR Spectrometer equipped with a 5 mm BBO probe head with Z-gradients, or a Bruker Avance 400 NMR spectrometer equipped with a 60 μl dual inverse flow probe head with Z-gradients, or a Bruker DPX400 NMR spectrometer equipped with a 4-nucleus probe head equipped with Z-gradients. Unless specifically noted in the examples, spectra were recorded at 400 MHz for proton and 100 MHz for carbon-13. The following reference signals were used: the middle line of DMSO-d₆ δ 2.50 (¹H), δ 39.51 (¹³C); the middle line of CD₃OD δ 3.31 (¹H) or δ 49.15 (¹³C); acetone-d₆ 2.04 (¹H), 206.5 (¹³C); and CDCl₃ δ 7.26 (¹H), the middle line of CDCl₃ δ 77.16 (¹³C) (unless otherwise indicated).

Mass spectra were recorded on a Waters LCMS consisting of an Alliance 2795 (LC), Waters PDA 2996, and ELS detector (Sedex 75) and a ZMD single quadrupole mass spectrometer. The mass spectrometer was equipped with an electrospray ion source (ES) operated in a positive or negative ion mode. The capillary voltage was 3 kV and cone voltage was 30 V. The mass spectrometer was scanned between m/z 100-600 with a scan time of 0.7 s. The column temperature was set to 40° C. The Diode Array Detector was scanned from 200-400 nm. The temperature of the ELS detector was adjusted to 40° C. and the pressure was set to 1.9 bar. For LC separation a linear gradient was applied starting at 100% A (A: 10 mM NH₄OAc in 5% MeCN) and ending at 100% B (B: MeCN) after four minutes. The column used was a X-Terra MS C8, 3.0×50; 3.5 μm (Waters) run at 1.0 mL/min.

HPLC analyses were performed on an Agilent HP1100 system consisting of G1379A Micro Vacuum Degasser, G1312A Binary Pump, G1367A Well plate auto-sampler, G1316A Thermostatted Column Compartment and G1315B Diode Array Detector. Column: X-Terra MS, Waters, 3.0×100 mm, 3.5 μm. The column temperature was set to 40° C. and the flow rate to 1.0 ml/min. The Diode Array Detector was scanned from 210-300 nm, step and peak width were set to 2 nm and 0.05 min, respectively. A linear gradient was applied, starting at 100% A (A: 10 mM NH₄OAc in 5% MeCN) and ending at 100% B (B: MeCN), in 6 min.

Preparative chromatography was run on a Waters autopurification HPLC with a diode array detector. Column: XTerra MS C8, 19×300 mm, 10 μm. Narrow gradients with MeCN/(95:5 0.1M NH₄OAc:MeCN) were used at a flow rate of 20 ml/min. Alternatively, another column was used; Atlantis C18 19×100 mm, 5 μm column. Gradient with acetonitrile/0.1M ammonium acetate in 5% acetonitrile in MilliQ Water, run from 0% to 35-50% acetonitrile, in 15 min. Flow rate: 15 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Narrow gradients with MeCN/0.1% trifluoroacetic acid in MilliQ Water were used at a flow rate of 10 ml/min.

The following abbreviations have been used:

aq. aqueous;
DMSO dimethylsulfoxide;
r.t. room temperature.

Starting materials used were either available from commercial sources or prepared according to literature procedures and had experimental data in accordance with those reported.

### EXAMPLES

The present invention is further illustrated by the following non-limiting examples.

### Example 1

### 3-(3-chlorophenyl)-2-thioxo-1,2,3,7-tetrahydro-6H-purin-6-one

### (a) 6-Amino-1-(3-chlorophenyl)-5-nitroso-2-thioxo-2,3-dihydropyrimidin-4(1H)-one

6-Amino-1-(3-chlorophenyl)-2-thioxo-2,3-dihydropyrimidin-4(1H)-one (Ganaphthi et al., Proceedings A., Chemical Sciences, 1953, 37A, 652-9) (1.1 g, 4.4 mmol) was dissolved in acetic acid (10% aq., 15 mL) and was heated at 75° C. for 20 minutes. Sodium nitrite (0.34 g, 4.9 mmol), dissolved in water (1.5 mL), was added and heating was continued for another 85 minutes. After cooling to r.t. the solid was collected by filtration, washed with water and dried to give the title compound (1.2 g, 92%) as a green solid. The crude product was used in the next step without further purification.

¹H NMR (DMSO-d₆) δ ppm 13.03-12.82 (m, 2H), 8.10 (br s, 1H), 7.61-7.55 (m, 3H), 7.42-7.40 (m, 1H); MS (ESI) m/z 283 (M+1).

### (b) 5,6-Diamino-1-(3-chlorophenyl)-2-thioxo-2,3-dihydropyrimidin-4(1H)-one

6-Amino-1-(3-chlorophenyl)-5-nitroso-2-thioxo-2,3-dihydropyrimidin-4(1H)-one (1.2 g, 4.1 mmol, obtained from Example 1(a)) was suspended in water (10 mL) and ammonia (32% aq., 10 mL) was added. The reaction mixture was heated at 75° C. and sodium dithionite (1.8 g, 10.2 mmol) was added and stirring was continued at 75° C. for 20 minutes, and then stirred at r.t. for 45 minutes. After adjusting the solution to neutral pH with 1M hydrochloric acid, the precipitated solid was collected by filtration, washed with water and dried, giving the title compound (0.92 g, 85%). The crude product was used in the next step without further purification.

¹H NMR (DMSO-d₆) δ ppm 12.07 (br s, 1H), 7.55-7.54 (m, 2H), 7.46 (m, 1H), 7.29-7.24 (m, 1H), 5.52 (s, 2H), 3.49 (s, 2H); MS (ESI) m/z 269 (M+1).

### (c) 3-(3-chlorophenyl)-2-thioxo-1,2,3,7-tetrahydro-6H-purin-6-one

5,6-Diamino-1-(3-chlorophenyl)-2-thioxo-2,3-dihydropyrimidin-4(1H)-one (0.92 g, 3.4 mmol, obtained from Example 1(b)) in formic acid (3.5 mL) was heated at 70° C. for 30 minutes. The excess of formic acid was evaporated in vacuo. Sodium hydroxide (10% aq., 7 mL) was added to the residue and the reaction mixture was heated at 70° C. for 85 minutes. The mixture was diluted with water and neutralized using 1M hydrochloric acid. The precipitated solid was collected by filtration, washed with water and dried. The crude product was purified by recrystallization from ethanol/water (80 mL/5 mL) followed by preparative HPLC, giving the title compound (0.32 g, 34%) as a solid.

¹H NMR (DMSO-d₆) δ ppm 13.86 (br s, 1H), 12.65 (br s, 1H), 8.01 (s, 1H), 7.56-7.55 (m, 3H), 7.40-7.36 (m, 1H); ¹³C NMR (DMSO-d₆) δ ppm 174.9, 152.9, 150.1, 141.0, 139.7, 133.0, 130.6, 129.2, 128.8, 128.1, 110.6; MS (ESI) m/z 279 (M+1).

### Example 2

### 3-(3-ethylphenyl)-2-thioxo-1,2,3,7-tetrahydro-6H-purin-6-one

### (a) N-(3-Ethylphenyl)thiourea

3-Ethylphenyl isothiocyanate (1.5 g, 9.2 mmol) was added, dropwise, to 7M ammonia in methanol (7 mL) at r.t. The reaction mixture was stirred at 40° C. for 90 minutes. After cooling to r.t. the solvent was removed in vacuo. Water was added and after some stirring a solid precipitated which was collected, washed with water and dried, giving the title compound (1.4 g, 87%). The crude product was used in the next step without further purification.

¹H NMR (DMSO-d₆) δ ppm 9.61 (s, 1H), 7.37 (br s, 2H), 7.25-7.21 (m, 3H), 6.98-6.96 (m, 1H), 2.58 (q, J=7.8 Hz, 2H), 1.17 (t, J=7.5 Hz, 3H); MS (ESI) m/z 181 (M+1).

### (b) 6-Amino-1-(3-ethylphenyl)-2-thioxo-2,3-dihydropyrimidin-4(1H)-one

Sodium ethoxide (21 wt % in ethanol, 9 mL) was added to a suspension of N-(3-ethylphenyl)thiourea (1.4 g, 7.7 mmol, obtained from Example 2(a)) in ethanol (9 mL). Ethyl cyanoacetate (0.65 g, 5.8 mmol) was added and the resulting mixture was stirred under reflux. Additional ethyl cyanoacetate (totally 1.6 g, 14.1 mmol) were added in portions during 6.5 h until the reaction had come to completion. After cooling to r.t. the reaction mixture was diluted with water (30 mL) and neutralized with 2M sulfuric acid. The precipitated solid was collected by filtration, washed with water and dried, giving the title compound (1.1 g, 57%). The crude product was used in the next step without further purification.

¹H NMR (DMSO-d₆) δ ppm 11.89 (br s, 1H), 7.45-7.41 (m, 1H), 7.33-7.31 (m, 1H), 7.11-7.07 (m, 2H), 6.15 (br s, 2H), 4.97 (s, 1H), 2.66 (q, J=7.6 Hz, 2H), 1.20 (t, J=7.5 Hz, 3H); MS (ESI) m/z 248 (M+1).

### (c) 6-Amino-1-(3-ethylphenyl)-5-nitroso-2-thioxo-2,3-dihydropyrimidin-4(1H)-one

6-Amino-1-(3-ethylphenyl)-2-thioxo-2,3-dihydropyrimidin-4(1H)-one (0.50 g, 2.0 mmol, obtained from Example 2(b)) was dissolved in acetic acid (50% aq.) and heated at 65° C. Sodium nitrite (0.15 g, 2.2 mmol), dissolved in water (1 mL), was added dropwise. Heating was continued for another 15 minutes. After cooling to r.t. the reaction mixture was diluted with water (20 mL). The precipitated solid was collected by filtration, washed with water and dried, giving the title compound (0.38 g, 68%). The crude product was used in the next step without further purification.

¹H NMR (DMSO-d₆) δ ppm 12.88 (br s, 2H), 7.71 (br s, 1H), 7.50-7.46 (m, 1H), 7.39-7.37 (m, 1H), 7.25 (s, 1H), 7.22-7.20 (m, 1H), 2.66 (q, J=7.5 Hz, 2H), 1.20 (t, J=7.5 Hz, 3H); MS (ESI) m/z 277 (M+1).

### (d) 5,6-Diamino-1-(3-ethylphenyl)-2-thioxo-2,3-dihydropyrimidin-4(1H)-one

6-Amino-1-(3-ethylphenyl)-5-nitroso-2-thioxo-2,3-dihydropyrimidin-4(1H)-one (0.38 g, 1.4 mmol, obtained from Example 2(c)) was suspended in water (4 mL) and ammonia (32% aq., 4 mL) was added. Sodium dithionite (0.60 g, 3.45 mmol) was added. The reaction mixture was stirred at r.t. for 1 h. After adjusting the solution to neutral pH with 2M sulfuric acid, the precipitated solid was collected by filtration, washed with water and dried, giving the title compound (0.25 g, 70%). The crude product was used in the next step without further purification.

¹H NMR (DMSO-d₆) δ ppm 7.47-7.42 (m, 1H), 7.34-7.32 (m, 1H), 7.10-7.06 (m, 2H), 5.31 (s, 2H), 2.66 (q, J=7.5 Hz, 2H), 1.21 (t, J=7.5 Hz, 3H); MS (ESI) m/z 263 (M+1).

### (e) 3-(3-ethylphenyl)-2-thioxo-1,2,3,7-tetrahydro-6H-purin-6-one

5,6-Diamino-1-(3-ethylphenyl)-2-thioxo-2,3-dihydropyrimidin-4(1H)-one (0.25 g, 0.92 mmol, obtained from Example 2(d)) in formic acid (2 mL) was heated at 70° C. for 30 minutes. Excess of formic acid was evaporated in vacuo. Sodium hydroxide (10% aq., 2 mL) was added to the residue and the reaction mixture was heated at 70° C. for 50 minutes. The mixture was diluted with water (10 mL) and neutralized using 2M sulfuric acid. The precipitated solid was collected by filtration, washed with water and dried. The crude product was purified by preparative HPLC, giving the title compound (0.068 g, 27%) as a solid.

¹H NMR (DMSO-d₆) δ ppm 13.84 (s, 1H), 12.60 (s, 1H), 7.99 (s, 1H), 7.44-7.40 (m, 1H), 7.32-7.30 (m, 1H), 7.18-7.14 (m, 2H), 2.66 (q, J=7.5 Hz, 2H), 1.20 (t, J=7.5 Hz, 3H);

¹³C NMR (DMSO-d₆) δ ppm 174.9, 152.9, 150.4, 144.8, 141.9, 138.6, 129.0, 128.0, 126.1, 110.9, 27.8, 15.2; MS (ESI) m/z 273 (M+1).

### Example 3

### 2-thioxo-3-[3-(trifluoromethoxy)phenyl]-1,2,3,7-tetrahydro-6H-purin-6-one

### (a) 6-Amino-2-thioxo-1-[3-(trifluoromethoxy)phenyl]-2,3-dihydropyrimidin-4(1H)-one

Sodium ethoxide (21 wt % in ethanol, 5 mL) and ethyl cyanoacetate (0.69 g, 6.1 mmol) were added to a solution of N-[3-(trifluoromethoxy)phenyl]thiourea (Jimonet, P. et al, J. Med. Chem. 1999, 15, 2828-2843) (1.2 g, 5.1 mmol) in absolute ethanol (5 mL). The mixture was stirred under reflux. Additional ethyl cyanoacetate (totally 0.51 g, 4.5 mmol) were added in portions during 4 h until the reaction had come to completion. After cooling to r.t. the reaction mixture was diluted with water (20 mL) and neutralized with 2M sulfuric acid. The precipitated solid was collected by filtration, washed with water and dried, giving the title compound (1.3 g, 82%). The crude product was used in the next step without further purification.

¹H NMR (DMSO-d₆) δ ppm 12.0 (s, 1H), 7.66-7.62 (m, 1H), 7.49-7.46 (m, 1H), 7.43 (m, 1H), 7.35-7.33 (m, 1H), 6.34 (s, 2H), 4.97 (s, 1H); MS (ESI) m/z 304 (M+1).

### (b) 6-Amino-5-nitroso-2-thioxo-1-[3-(trifluoromethoxy)phenyl]-2,3-dihydropyrimidin-4(1H)-one

6-Amino-2-thioxo-1-[3-(trifluoromethoxy)phenyl]-2,3-dihydropyrimidin-4(1H)-one (0.60 g, 2.0 mmol, obtained from Example 3(a)) was suspended in acetic acid (50% aq., 6 mL) and heated at 75° C. Sodium nitrite (0.15 g, 2.2 mmol), dissolved in water (1 mL), was added dropwise. Heating was continued for another 30 minutes. The reaction mixture was diluted with water (20 mL) and the mixture was stirred at r.t. for 1 h. The precipitated solid was collected by filtration, washed with water and dried, giving the title compound (0.48 g, 73%). The crude product was used in the next step without further purification.

¹H NMR (DMSO-d₆) δ ppm 12.94 (br s, 2H), 8.10 (br s, 1H), 7.72-7.68 (m, 1H), 7.56-7.53 (m, 2H), 7.48-7.46 (m, 1H); MS (ESI) m/z 333 (M+1).

### (c) 5,6-Diamino-2-thioxo-1-[3-(trifluoromethoxy)phenyl]-2,3-dihydropyrimidin-4(1H)-one

Sodium dithionite (0.73 g, 4.2 mmol) was added to a suspension of 6-amino-5-nitroso-2-thioxo-1-[3-(trifluoromethoxy)phenyl]-2,3-dihydropyrimidin-4(1H)-one (0.47 g, 1.4 mmol, obtained from Example 3(b)) in water (4 mL) and ammonia (32% aq., 4 mL). The reaction mixture was stirred at r.t. for 2 h. After adjusting the solution to neutral pH with 2M sulfuric acid, the precipitated solid was collected by filtration, washed with water and dried, giving the title compound (0.35 g, 78%). The crude product was used in the next step without further purification.

¹H NMR (DMSO-d₆) δ ppm 7.67-7.63 (m, 1H), 7.49-7.47 (m, 2H), 7.39 (m, 1H), 7.34-7.32 (m, 1H), 5.52 (s, 2H); MS (ESI) m/z 319 (M+1).

### (d) 2-thioxo-3-[3-(trifluoromethoxy)phenyl]-1,2,3,7-tetrahydro-6H-purin-6-one

5,6-Diamino-2-thioxo-1-[3-(trifluoromethoxy)phenyl]-2,3-dihydropyrimidin-4(1H)-one (0.35 g, 1.1 mmol, obtained from Example 3(c)) in formic acid (aq., 3 mL) was heated at 70° C. for 25 minutes. The excess formic acid was evaporated in vacuo. Sodium hydroxide (10% aq., 3 mL) was added to the residue and the reaction mixture was heated at 70° C. for 1 h. The mixture was diluted with water (10 mL) and neutralized using 2M sulfuric acid. The precipitated solid was collected by filtration, washed with water and dried. The crude product was purified by preparative HPLC, giving the title compound (0.043 g, 12%) as a solid.

¹H NMR (DMSO-d₆) δ ppm 13.89 (s, 1H), 12.69 (s, 1H), 8.02 (s, 1H), 7.68-7.64 (m, 1H), 7.51-7.44 (m, 3H); ¹³C NMR (DMSO-d₆) δ ppm 175.0, 152.9, 150.2, 148.5, 141.1, 139.8, 130.7, 128.6, 122.3, 121.4, 118.7, 110.6; MS (ESI) m/z 329 (M+1).

### Example 4

### 3-(4-chlorophenyl)-2-thioxo-1,2,3,7-tetrahydro-6H-purin-6-one

### (a) 6-Amino-1-(4-chlorophenyl)-5-nitroso-2-thioxo-2,3-dihydropyrimidin-4(1H)-one

6-Amino-1-(4-chlorophenyl)-2-thioxo-2,3-dihydro-4(1H)-pyrimidinone (Ganaphthi et al., Proceedings A., Chemical Sciences, 1953, 37A, 652-9) (0.60 g, 2.4 mmol) was suspended in acetic acid (10% aq., 5 mL), and heated at 75° C., before dropwise addition of sodium nitrite (0.24 g, 3.6 mmol) dissolved in water (5 mL), during 15 minutes. More water (2 mL) was added to facilitate stirring and heating was continued for 1 h. The reaction mixture was diluted with water (20 mL) and the solid was collected by filtration, washed with water and dried to give the title compound (0.60 g, 89%) as a green solid. The crude product was used in the next step without further purification.

¹H NMR (DMSO-d₆) δ ppm 12.88 (br s, 2H), 8.04 (br s, 1H), 7.65-7.61 (m, 2H), 7.46-7.43 (m, 2H); MS (ESI) m/z 283 (M+1).

### (b) 5,6-Diamino-1-(4-chlorophenyl)-2-thioxo-2,3-dihydropyrimidin-4(1H)-one

6-Amino-1-(4-chlorophenyl)-5-nitroso-2-thioxo-2,3-dihydropyrimidin-4(1H)-one (0.60 g, 2.1 mmol, obtained from Example 4(a)) was suspended in water (8 mL) and ammonia (32% aq., 8 mL). Sodium dithionite (1.1 g, 6.3 mmol) was added. The resulting suspension was stirred at r.t. for 1 h. The reaction mixture was neutralized using 2M sulfuric acid. The solid was collected by filtration, washed with water and dried, giving the title compound (0.46 g, 82%) as a yellow solid. The crude product was used in the next step without further purification.

¹H NMR (DMSO-d₆) δ ppm 11.79 (br s, 1H), 7.60-7.56 (m, 2H), 7.33-7.29 (m, 2H), 5.53 (s, 2H), 3.49 (s, 2H); MS (ESI) m/z 269 (M+1).

### (c) 3-(4-chlorophenyl)-2-thioxo-1,2,3,7-tetrahydro-6H-purin-6-one

5,6-Diamino-1-(4-chlorophenyl)-2-thioxo-2,3-dihydropyrimidin-4(1H)-one (0.46 g, 1.7 mmol, obtained from Example 4(b)) in formic acid (3 mL) was heated at 70° C. for 30 minutes. The excess formic acid was evaporated in vacuo. Sodium hydroxide (10% aq., 6 mL) was added to the residue and the reaction mixture was heated at 70° C. for 2 h. After dilution with water (10 mL), the reaction mixture was neutralized using 2M sulfuric acid. The precipitated solid was collected by filtration, washed with water and dried. The crude product was purified by preparative HPLC, giving the title compound (0.26 g, 55%) as a solid.

¹H NMR (DMSO-d₆) δ ppm 13.87 (br s, 1H), 12.67 (s, 1H), 8.01 (s, 1H), 7.61-7.57 (m, 2H), 7.44-7.39 (m, 2H); ¹³C NMR (DMSO-d₆) δ ppm 175.0, 152.9, 150.2, 141.1, 137.5, 133.3, 131.0, 129.2, 110.6; MS (ESI) m/z 279 (M+1).

### Example 5

### 3-(3,5-dichlorophenyl)-2-thioxo-1,2,3,7-tetrahydro-6H-purin-6-one

### (a) 6-Amino-1-(3,5-dichlorophenyl)-2-thioxo-2,3-dihydropyrimidin-4(1H)-one

A suspension of 3,5-dichlorophenylthiourea (1.5 g, 6.8 mmol) in absolute ethanol (5 mL) and sodium ethoxide (21 wt % in ethanol, 5 mL) was heated at reflux. Ethyl cyanoacetate (1.5 g, 13.6 mmol) dissolved in ethanol (5 mL) was added during 30 minutes. Heating was continued for 1 h. Additional ethyl cyanoacetate (0.5 g) in ethanol (1 mL) was added and heating was continued for another 1 h. After cooling to r.t. the reaction mixture was diluted with water (20 mL) and neutralized using 2M sulfuric acid. The solid was collected by filtration, washed with water and dried, giving the title compound (1.5 g, 75%). The crude product was used in the next step without further purification.

¹H NMR (DMSO-d₆) δ ppm 11.98 (br s, 1H), 7.70 (t, J=1.8 Hz, 1H), 7.46 (m, 2H), 6.27 (br s, 2H), 4.92 (s, 1H), MS (ESI) m/z 289 (M+1).

### (b) 6-Amino-1-(3,5-dichlorophenyl)-5-nitroso-2-thioxo-2,3-dihydropyrimidin-4(1H)-one

A suspension of 6-amino-1-(3,5-dichlorophenyl)-2-thioxo-2,3-dihydropyrimidin-4(1H)-one (0.60 g, 2.1 mmol, obtained from Example 5(a)) in acetic acid (4 mL) was heated at 75° C. for 5 minutes. Sodium nitrite (0.22 g, 3.1 mmol), dissolved in water (4 mL), was added dropwise during 10 minutes. Heating was continued for 1 h. The reaction mixture was diluted with water (30 mL) and cooled to r.t. The solid was collected by filtration, washed with water and dried, giving the title compound (0.54 g, 82%). The crude product was used in the next step without further purification.

¹H NMR (DMSO-d₆) δ ppm 13.0 (s, 1H), 12.87 (s, 1H), 8.30 (s, 1H), 7.82 (s, 1H), 7.62 (s, 2H); MS (ESI) m/z 318 (M+1).

### (c) 5,6-Diamino-1-(3,5-dichlorophenyl)-2-thioxo-2,3-dihydropyrimidin-4(1H)-one

Sodium dithionite (0.74 g, 4.3 mmol) was added to a suspension of 6-amino-1-(3,5-dichlorophenyl)-5-nitroso-2-thioxo-2,3-dihydropyrimidin-4(1H)-one (0.54 g, 1.7 mmol, obtained from Example 5(b)) in water (6 mL) and ammonia (32% aq., 6 mL). The resulting suspension was stirred at r.t. for 3 h. The reaction mixture was diluted with water (15 mL) and then neutralized using 2M sulfuric acid. The solid was collected by filtration, washed with water and dried, giving the title compound (0.45 g, 86%) as a yellow solid. The crude product was used in the next step without further purification.

¹H NMR (DMSO-d₆) δ ppm 7.73 (t, J=2.0 Hz, 1H), 7.50 (d, J=2.0 Hz, 2H), 5.67 (s, 3H), 3.51 (br s, 2H); MS (ESI) m/z 304 (M+1).

### (d) 3-(3,5-dichlorophenyl)-2-thioxo-1,2,3,7-tetrahydro-6H-purin-6-one

5,6-Diamino-1-(3,5-dichlorophenyl)-2-thioxo-2,3-dihydropyrimidin-4(1H)-one (0.44 g, 1.5 mmol, obtained from Example 5(c)) in formic acid (3 mL) was heated at 70° C. for 20 minutes. The excess formic acid was evaporated in vacuo. Sodium hydroxide (10% aq., 4 mL) was added to the residue and the reaction mixture was heated at 70° C. for 1.5 h. After dilution with water (10 mL) the reaction mixture was filtered to remove insoluble material. The filtrate was neutralized using 2M sulfuric acid. The precipitated solid was collected by filtration, washed with water and dried. The crude product was purified by preparative HPLC, giving the title compound (0.21 g, 47%) as a solid.

¹H NMR (DMSO-d₆) δ ppm 13.90 (br s, 1H), 12.73 (br s, 1H), 8.03 (s, 1H), 7.78 (t, J=1.9 Hz, 1H), 7.62 (d, J=1.8 Hz, 2H), ¹³C NMR (DMSO-d₆) δ ppm 174.8, 152.9, 150.0, 141.1, 140.6, 134.1, 128.7, 128.6, 110.6; MS (ESI) m/z 314 (M+1).

### Example 6

### 3-(6-Oxo-2-thioxo-1,2,6,7-tetrahydro-3H-purin-3-yl)benzonitrile

### (a) 3-(6-Amino-4-oxo-2-thioxo-3,4-dihydropyrimidin-1(2H)-yl)benzonitrile

A suspension of N-(3-cyanophenyl)thiourea (Dyson, G. M. et al, J. Chem. Soc. 1927, 436-445) (1.0 g, 5.6 mmol) in absolute ethanol (5 mL) was heated at 75° C. Ethyl cyanoacetate (1.3 g, 11.3 mmol) dissolved in ethanol (6 mL), and sodium ethoxide (1M in ethanol, 7.3 mL) was added during 1 h. Heating was continued for 1.5 h. The reaction mixture was poured into water (100 mL) and was neutralized using 2M sulfuric acid. The mixture was stirred for 0.5 h and the precipitated product was collected by filtration, washed with water and dried, giving the title compound (0.56 g, 40%). The crude product was used in the next step without further purification.

¹H NMR (DMSO-d₆) δ ppm 12.08 (s, 1H), 7.97-7.94 (m, 2H), 7.73-7.66 (m, 2H), 6.41 (s, 2H), 4.97 (d, J=1.8 Hz, 1H); MS (ESI) m/z 245 (M+1).

### (b) 3-(6-Amino-5-nitroso-4-oxo-2-thioxo-3,4-dihydropyrimidin-1(2H)-yl)benzonitrile

To a suspension of 3-(6-amino-4-oxo-2-thioxo-3,4-dihydropyrimidin-1(2H)-yl)benzonitrile (0.47 g, 1.9 mmol, obtained from Example 6(a)) in acetic acid (50%, 3 mL) was added sodium nitrite (0.16 g, 2.3 mmol). The resulting slurry was stirred at r.t. for 3 h. After diluting the reaction mixture with water (10 mL), the solid was collected by filtration, washed with water and dried, giving the title compound (0.49 g, 93%). The crude product was used in the next step without further purification.

¹H NMR (DMSO-d₆) δ ppm 13.03 (s, 1H), 12.86 (s, 1H), 8.19 (br s, 1H), 8.04-8.02 (m, 1H), 7.97 (s, 1H), 7.81-7.78 (m, 2H); MS (ESI) m/z 274 (M+1).

### (c) 3-(5,6-Diamino-4-oxo-2-thioxo-3,4-dihydropyrimidin-1(2H)-yl)benzonitrile

Ammonia (32% aq., 3 mL) was added to 3-(6-amino-5-nitroso-4-oxo-2-thioxo-3,4-dihydropyrimidin-1(2H)-yl)benzonitrile (0.48 g, 1.8 mmol, obtained from Example 6(b)) in water (3 mL). Sodium dithionite (0.76 g, 4.4 mmol) was added during 5 minutes. The resulting suspension was stirred at r.t. for 1 h. After neutralization, using 2M sulfuric acid, the solid was collected by filtration, washed with water and dried, giving the title compound (0.34 g, 75%) as an orange solid. The crude product was used in the next step without further purification.

¹H NMR (DMSO-d₆) δ ppm 11.24 (br s, 1H), 7.96-7.92 (m, 2H), 7.72 (t, J=7.9 Hz, 1H), 7.66-7.64 (m, 1H), 5.60 (s, 2H), 3.54 (br s, 2H); MS (ESI) m/z 260 (M+1).

### (d) 3-(6-Oxo-2-thioxo-1,2,6,7-tetrahydro-3H-purin-3-yl)benzonitrile

A mixture of 3-(5,6-diamino-4-oxo-2-thioxo-3,4-dihydropyrimidin-1(2H)-yl)benzonitrile (0.34 g, 1.3 mmol, obtained from Example 6(c)) and formamidine acetate (0.22 g, 2.1 mmol) in DMSO (2 mL) was heated at 70° C. for 1 h. The crude product in DMSO was purified by preparative HPLC, giving the title compound (0.11 g, 32%) as a solid.

¹H NMR (DMSO-d₆) δ ppm 13.92 (s, 1H), 12.76 (s, 1H), 8.04 (s, 1H), 8.02 (t, J=1.8 Hz, 1H), 7.99-7.96 (m, 1H), 7.81-7.74 (m, 2H); ¹³C NMR (DMSO-d₆) δ ppm 175.0, 152.9, 150.1, 141.1, 139.3, 134.7, 133.2, 132.7, 130.6, 118.0, 112.0, 110.7; MS (ESI) m/z 270 (M+1).

### Example 7

### 3-(4-methoxyphenyl)-2-thioxo-1,2,3,7-tetrahydro-6H-purin-6-one

### (a) 6-Amino-1-(4-methoxyphenyl)-5-nitroso-2-thioxo-2,3-dihydropyrimidin-4(1H)-one

6-Amino-1-(4-methoxyphenyl)-2-thioxo-2,3-dihydropyrimidin-4(1H)-one (Ganaphthi et al., Proceedings A., Chemical Sciences, 1953, 37A, 652-9) (0.50 g, 2.0 mmol) in formic acid (10% aq., 12 mL) was heated at 85° C. for 10 minutes. Sodium nitrite (0.15 g, 2.2 mmol) dissolved in water (1 mL), was added dropwise during 10 minutes. Heating with vigorous stirring was continued for 40 minutes. The reaction mixture was diluted with water (15 mL) and cooled to r.t. The solid was collected by filtration, washed with water and dried, giving the title compound (0.47 g, 84%) as a green solid. The crude product was used in the next step without further purification.

¹H NMR (DMSO-d₆) δ ppm 12.87-12.85 (m, 2H), 7.82 (s, 1H), 7.33-7.29 (m, 2H), 7.12-7.08 (m, 2H), 3.82 (s, 3H); MS (ESI) m/z 279 (M+1).

### (b) 5,6-Diamino-1-(4-methoxyphenyl)-2-thioxo-2,3-dihydropyrimidin-4(1H)-one

Ammonia (32% aq., 5 mL) was added to a suspension of 6-amino-1-(4-methoxyphenyl)-5-nitroso-2-thioxo-2,3-dihydropyrimidin-4(1H)-one (0.47 g, 1.7 mmol obtained from Example 7(a)) in water (5 ml) and the resulting mixture was heated at 75° C. Sodium dithionite (0.73 g, 4.2 mmol) was added in portions during 3 minutes. Heating was continued for another 15 minutes and then the reaction mixture was stirred at r.t. for 45 minutes. After neutralization with 1M hydrochloric acid the solid was collected by filtration, washed with water and dried, giving the title compound (0.37 g, 83%) as a solid. The crude product was used in the next step without further purification.

¹H NMR (DMSO-d₆) δ ppm 11.85 (br s, 1H), 7.19-7.15 (m, 2H), 7.08-7.04 (m, 2H), 5.40 (s, 2H), 4.36 (s, 2H), 3.82 (s, 3H); MS (ESI) m/z 265 (M+1).

### (c) 3-(4-methoxyphenyl)-2-thioxo-1,2,3,7-tetrahydro-6H-purin-6-one

5,6-Diamino-1-(4-methoxyphenyl)-2-thioxo-2,3-dihydropyrimidin-4(1H)-one (0.37 g, 1.4 mmol, obtained from Example 7(b)) in formic acid (1 mL) was heated at 70° C. for 20 minutes. The excess of formic acid was evaporated in vacuo. Sodium hydroxide (10% aq., 3 mL) was added to the residue and the reaction mixture was heated at 70° C. for 70 minutes. After dilution with water (10 mL) the reaction mixture was neutralized using 1M hydrochloride. The precipitated solid was collected by filtration, washed with water and dried. The crude product was purified by preparative HPLC, giving the title compound (0.062 g, 16%) as a solid.

¹H NMR (DMSO-d₆) δ ppm 13.79 (br s, 1H), 12.55 (s, 1H), 7.98 (s, 1H), 7.27-7.23 (m, 2H), 7.06-7.02 (m, 2H), 3.82 (s, 3H); ¹³C NMR (DMSO-d₆) δ ppm 175.3, 159.0, 152.8, 141.0, 131.3, 129.9, 114.2, 55.3; MS (ESI) m/z 275 (M+1).

### Example 8

### 3-quinolin-3-yl-2-thioxo-1,2,3,7-tetrahydro-6H-purin-6-one

### (a) 6-Amino-1-quinolin-3-yl-2-thioxo-2,3-dihydropyrimidin-4(1H)-one

N-(3-Quinolinyl)thiourea (2.0 g, 9.8 mmol) was suspended in absolute ethanol (5 mL) and sodium ethoxide (21 wt % in ethanol, 5 mL) was added followed by ethyl cyanoacetate (1.3 g, 11.8 mmol). The reaction mixture was heated at reflux and additional ethanol (5 mL) was added to facilitate stirring. Additional ethyl cyanoacetate (totally 3.1 g) and sodium ethoxide (21 wt % in ethanol, 7.5 mL) were added during 24 h until the reaction had come to completion. The reaction mixture was cooled to r.t., diluted with water (20 mL) and neutralized using 2M sulfuric acid. The precipitated product was collected by filtration, washed with water and dried to give the title compound (1.8 g, 66%) as a solid. The crude product was used in the next step without further purification.

¹H NMR (DMSO-d₆) δ ppm 12.14 (s, 1H), 8.74 (d, J=2.6 Hz, 1H), 8.42 (d, J=2.2 Hz, 1H), 8.09 (d, J=8.6 Hz, 1H), 8.05 (d, J=7.5 Hz, 1H), 7.88-7.84 (m, 1H), 7.71-7.66 (m, 1H), 6.58 (s, 2H), 5.03 (d, J=1.5 Hz, 1H); MS (ESI) m/z 271 (M+1).

### (h) 6-Amino-5-nitroso-1-quinolin-3-yl-2-thioxo-2,3-dihydropyrimidin-4(1H)-one

6-Amino-1-quinolin-3-yl-2-thioxo-2,3-dihydropyrimidin-4(1H)-one (0.75 g, 2.8 mmol obtained from Example 8(a)) was dissolved in acetic acid (90% aq., 15 mL) and was heated at 60° C. for 5 minutes. Sodium nitrite (0.21 g, 3.1 mmol), dissolved in water (1 mL), was added dropwise and heating was continued for 10 minutes. Additional water (2+2 mL) was added, to facilitate stirring, and additional sodium nitrite (0.060 g) in water (1 mL) was also added. After 1 h the reaction mixture was diluted with water (30 mL) and the solid was collected by filtration and dried at r.t. overnight. The solid was then suspended in acetic acid (90%, 7 mL), heated at 60° C. before addition of sodium nitrite (0.15 g) in water (1 mL). After heating for 40 minutes the reaction mixture was cooled to r.t., diluted with water (30 mL) and the solid was collected by filtration, washed with water and dried to give the title compound (0.54 g, 65%) as a solid. The crude product was used in the next step without further purification.

¹H NMR (DMSO-d₆) δ ppm 13.08 (s, 1H), 12.87 (s, 1H), 8.84 (s, J=2.3 Hz, 1H), 8.48 (m, 1H), 8.43 (s, 1H), 8.12 (d, J=8.6 Hz, 1H), 8.05 (d, J=8.0 Hz, 1H), 7.92-7.88 (m, 1H), 7.71 (t, J=7.6 Hz, 1H); MS (ESI) m/z 300 (M+1).

### (c) 5,6-Diamino-1-quinolin-3-yl-2-thioxo-2,3-dihydropyrimidin-4(1H)-one

A suspension of 6-amino-5-nitroso-1-quinolin-3-yl-2-thioxo-2,3-dihydropyrimidin-4(1H)-one (0.75 g, 4.3 mmol, obtained from Example 8(b)) in ammonia (32% aq., 5 mL) was diluted with water (5 mL) and the mixture was heated at 60° C. Sodium dithionite (0.75 g, 4.3 mmol) was added in small portions during 2 minutes and heating was continued for another 15 minutes and then the reaction mixture was stirred at r.t. for 1 h. After neutralization, with 2M sulfuric acid, the precipitated solid was collected by filtration, washed with water and dried, giving the title compound (0.37 g, 72%) as a solid. The crude product was used in the next step without further purification.

¹H NMR (DMSO-d₆) δ ppm 8.72 (d, J=2.5 Hz, 1H), 8.38 (d, J=2.5 Hz, 1H), 8.09 (d, J=8.3 Hz, 1H), 8.06 (d, J=7.5 Hz, 1H), 7.88-7.84 (m, 1H), 7.70-7.66 (m, 1H), 5.78 (s, 2H); MS (ESI) m/z 286 (M+1).

### (d) 3-quinolin-3-yl-2-thioxo-1,2,3,7-tetrahydro-6H-purin-6-one

5,6-Diamino-1-quinolin-3-yl-2-thioxo-2,3-dihydropyrimidin-4(1H)-one (0.37 g, 1.3 mmol, obtained from Example 8(c)) in formic acid (3 mL) was heated at 60° C. for 25 minutes. The excess formic acid was evaporated in vacuo. Sodium hydroxide (10% aq., 10 mL) was added to the residue and the reaction mixture was heated at 60° C. for 3 h. After dilution with water (10 mL) the reaction mixture was neutralized using 2M sulfuric acid. The precipitated solid was collected by filtration and purified by preparative HPLC, giving the title compound (0.025 g, 7%) as a yellow solid.

¹H NMR (DMSO-d₆) δ ppm 13.97 (s, 1H), 12.84 (s, 1H), 8.89 (d, J=2.5 Hz, 1H), 8.50 (d, J=2.0 Hz, 1H), 8.12 (d, J=8.6 Hz, 1H), 8.07 (d, J=8.3 Hz, 1H), 8.05 (s, 1H), 7.90-7.86 (m, 1H), 7.73-7.69 (m, 1H); ¹³C NMR (DMSO-d₆) δ ppm 175.4, 152.9, 150.8, 150.3, 146.7, 141.2, 135.5, 132.2, 130.5, 128.7, 128.4, 127.6, 127.3, 110.8; MS (ESI) m/z 296 (M+1).

### Example 9

### 3-(2,5-difluorophenyl)-2-thioxo-1,2,3,7-tetrahydro-6H-purin-6-one

### (a) 6-Amino-1-(2,5-difluorophenyl)-2-thioxo-2,3-dihydropyrimidin-4(1H)-one

A solution of 1-(2,5-difluorophenyl)-2-thiourea (0.27 g, 2.8 mmol) in ethanol (5 mL) was heated to 80° C. Sodium ethoxide (21 wt %, 2.1 mL, 5.5 mmol) and a solution of ethyl cyanoacetate (0.59 mL, 5.5 mmol) in ethanol (2 mL) was added, dropwise, over 45 minutes. The mixture was stirred under continued heating for 1.5 h and then at r.t. overnight. Additional sodium ethoxide (21 wt %, 1 mL) and ethyl cyanoacetate (0.29 mL, in 1 mL ethanol) were added during 1 h, and the reaction was kept at 80° C. for 3 h. After cooling to r.t. the reaction was partly concentrated. After addition of water (100 mL) the reaction was acidified with 2M sulphuric acid and kept in the refrigerator overnight. The precipitated solid was collected by filtration, washed with water and dried, giving the title compound (0.56 g, 80%) as an orange solid.

¹H NMR (DMSO-d₆) δ ppm 12.41 (s, 1H), 7.52-7.44 (m, 3H), 6.60 (s, 2H), 4.97 (s, 1H); MS (ESI) m/z 256 (M+1).

### (b) 5,6-Diamino-1-(2,5-difluorophenyl)-2-thioxo-2,3-dihydropyrimidin-4(1H)-one

To a solution of 6-amino-1-(2,5-difluorophenyl)-2-thioxo-2,3-dihydropyrimidin-4(1H)-one (0.68 g, 2.7 mmol, obtained from Example 9(a)) in acetic acid (5 mL) was added, dropwise over 5 minutes, sodium nitrite (0.20 g, 3.0 mmol) in water (1 mL). After stirring for 20 minutes sodium dithionite (1.4 g, 8.0 mmol) was added. After 30 minutes the reaction mixture was concentrated and water (50 mL) was added to the residue. The precipitated solid was collected by filtration, washed with water and dried, giving the title compound (0.28 g, 39%) as a solid.

¹H NMR (DMSO-d₆) δ ppm 12.39 (s, 1H), 7.50-7.43 (m, 3H), 5.78 (s, 2H), 3.57 (s, 2H); MS (ESI) m/z 271 (M+1).

### (c) 3-(2,5-difluorophenyl)-2-thioxo-1,2,3,7-tetrahydro-6H-purin-6-one

A solution of 5,6-diamino-1-(2,5-difluorophenyl)-2-thioxo-2,3-dihydropyrimidin-4(1H)-one (0.31 g, 1.1 mmol obtained from Example 9(b)) and formamidine acetate (0.18 g, 1.7 mmol) in DMSO (2 mL) was heated to 70° C. for 2 h. After cooling to r.t. the mixture was diluted with acetonitrile (2 mL) and the product mixture was purified by preparative HPLC, giving the title compound (0.16 g, 50%) as a solid.

¹H NMR (DMSO-d₆) δ ppm 13.90 (s, 1H), 12.76 (s, 1H), 7.99 (s, 1H), 7.51-7.36 (m, 3H);

¹³C NMR (DMSO-d₆) δ 175.2, 153.1, 150.0, 141.8, 110.6; MS (ESI) m/z 281 (M+1).

### Example 10

### 3-(3-fluorophenyl)-2-thioxo-1,2,3,7-tetrahydro-6H-purin-6-one

### (a) 6-Amino-1-(3-fluorophenyl)-2-thioxo-2,3-dihydropyrimidin-4(1H)-one

A solution of N-(3-fluorophenyl)thiourea (1.0 g, 5.9 mmol) and sodium ethoxide (1 M, 8.8 mL, 8.8 mmol) in absolute ethanol (3 mL) was heated to 90° C. A solution of ethyl cyanoacetate (1.3 mL, 12 mmol) in ethanol (5 mL) was added dropwise over 45 minutes. The mixture was stirred under continued heating for 3 h. After cooling to r.t. the reaction was concentrated to half its volume. Water (20 mL) was added and the mixture was neutralized with 2M sulphuric acid. The precipitated solid was collected by filtration, washed with water and dried, giving the title compound (1.23 g, 88%) as a white solid.

¹H NMR (DMSO-d₆) δ ppm 11.94 (s, 1H), 7.58-7.52 (m, 1H), 7.35-7.28 (m, 2H), 7.16-7.12 (m, 1H), 6.32 (s, 2H), 4.96 (s, 1H); MS (ESI) m/z 238 (M+1).

### (b) 6-Amino-1-(3-fluorophenyl)-5-nitroso-2-thioxo-2,3-dihydropyrimidin-4(1H)-one

A slurry of 6-amino-1-(3-fluorophenyl)-2-thioxo-2,3-dihydropyrimidin-4(1H)-one (0.60 g, 2.5 mmol, obtained from Example 10(a)) in acetic acid (50% aq., 4 mL) was heated at 75° C. for 5 minutes. Sodium nitrite (0.19 g, 2.8 mmol), dissolved in water (1 mL), was added during 10 minutes and heating was continued for another 4 hours. After cooling to r.t. the solid was collected by filtration, washed with water and dried to give the title compound (0.57 g, 85%) as a green solid. The crude product was used in the next step without further purification.

¹H NMR (DMSO-d₆) 8 ppm 12.93-12.91 (m, 2H), 8.02 (s, 1H), 7.64-7.59 (m, 1H), 7.42-7.38 (m, 2H), 7.30-7.28 (d, 1H); MS (ESI) m/z 267 (M+1).

### (c) 5,6-Diamino-1-(3-fluorophenyl)-2-thioxo-2,3-dihydropyrimidin-4(1H)-one

6-Amino-1-(3-fluorophenyl)-5-nitroso-2-thioxo-2,3-dihydropyrimidin-4(1H)-one (0.57 g, 2.1 mmol, obtained from Example 10(b)) was suspended in water (8 mL) and ammonia (28% aq., 5 mL) was added. The reaction mixture was heated at 75° C. and sodium dithionite (0.93 g, 5.4 mmol) was added in portions and stirring was continued at 75° C. for 15 minutes, and then stirred at r.t. for 1.5 hours. After adjusting the solution to neutral pH with 2M sulfuric acid, the precipitated solid was collected by filtration, washed with water and dried, giving the title compound (0.42 g, 78%). The crude product was used in the next step without further purification.

¹H NMR (DMSO-d₆) δ ppm 11.99 (s, 1H), 7.59-7.54 (m, 1H), 7.36-7.31 (m, 1H), 7.29-7.25 (m, 1H), 7.14-7.12 (m, 1H), 5.50 (s, 2H), 3.48 (s, 2H); MS (ESI) m/z 253 (M+1).

### (d) 3-(3-fluorophenyl)-2-thioxo-1,2,3,7-tetrahydro-6H-purin-6-one

A solution of 5,6-diamino-1-(3-fluorophenyl)-2-thioxo-2,3-dihydropyrimidin-4(1H)-one (0.41 g, 1.6 mmol, obtained from Example 10(c)) and formamidine acetate (0.34 g, 3.3 mmol) in DMSO (2 mL) was heated to 70° C. for 2 h. The crude product mixture was purified by preparative HPLC, giving the title compound (0.30 g, 71%) as a solid.

¹H NMR (DMSO-d₆) 8 ppm 13.88 (br s, 1H), 12.67 (br s, 1H), 8.01 (s, 1H), 7.60-7.54 (m, 1H), 7.36-7.32 (m, 2H), 7.25-7.23 (m, 1H); ¹³C NMR (DMSO-d₆) δ ppm 174.9, 162.1 (d), 152.9, 150.2, 141.1, 139.9 (d), 130.7, 125.5, 116.6 (d), 115.8 (d), 110.6; MS (ESI) m/z 263 (M+1).

### Example 11

### 3-(2-chlorophenyl)-2-thioxo-1,2,3,7-tetrahydro-6H-purin-6-one

### (a) 6-Amino-1-(2-chlorophenyl)-5-nitroso-2-thioxo-2,3-dihydropyrimidin-4(1H)-one

6-Amino-1-(2-chlorophenyl)-2-thioxo-2,3-dihydropyrimidin-4(1H)-one (Ganaphthi et al, Proceedings A., Chemical Sciences, 1953, 37A, 652-9) (0.81 g, 3.2 mmol) was suspended in acetic acid (40% aq., 13 mL) and was heated at 75° C. for 5 minutes. Sodium nitrite (0.33 g, 4.8 mmol), dissolved in water (3 mL), was added during 1 hour and heating was continued for another 0.5 hour. Additional sodium nitrite (totally 0.88 g dissolved in 8 mL water) was added during 8 hours until the reaction was complete. During the last 3 hours, the temperature was raised to 90° C. The reaction mixture was poured onto ice and the solid was collected by filtration, washed with water and dried to give the title compound (0.39 g, 43%) as a purple solid. The crude product was used in the next step without further purification.

¹H NMR (DMSO-d₆) 8 ppm 13.10 (br s, 1H), 12.78 (br s, 1H), 8.35 (br s, 1H), 7.71-7.69 (m, 1H), 7.61-7.53 (m, 3H); MS (ESI) m/z 283 (M+1).

### (b) 5,6-Diamino-1-(2-chlorophenyl)-2-thioxo-2,3-dihydropyrimidin-4(1H)-one

6-Amino-1-(2-chlorophenyl)-5-nitroso-2-thioxo-2,3-dihydropyrimidin-4(1H)-one (0.39 g, 1.4 mmol obtained from Example 11(a)) was suspended in water (4 mL) and ammonia (28% aq., 4 mL) was added. The reaction mixture was heated at 75° C. and sodium dithionite (0.60 g, 3.5 mmol) was added and stirring was continued at 75° C. for 15 minutes, and then stirred at r.t. for 1 hour. After adjusting the solution to neutral pH with 2M sulfuric acid, the precipitated solid was collected by filtration, washed with water and dried, giving the title compound (0.30 g, 81%). The crude product was used in the next step without further purification.

¹H NMR (DMSO-d₆) δ ppm 12.16 (br s, 1H), 7.66-7.63 (m, 1H), 7.54-7.43 (m, 3H), 5.55 (s, 2H), 3.50 (s, 2H); MS (ESI) m/z 269 (M+1).

### (c) 3-(2-chlorophenyl)-2-thioxo-1,2,3,7-tetrahydro-6H-purin-6-one

A solution of 5,6-diamino-1-(3-chlorophenyl)-2-thioxo-2,3-dihydropyrimidin-4(1H)-one (0.30 g, 1.1 mmol) and formamidine acetate (0.23 g, 2.2 mmol, obtained from Example 11(b)) in DMSO (2 mL) was heated to 70° C. for 2 h. The crude product mixture was purified by preparative HPLC, giving the title compound (0.17 g, 55%) as a solid.

¹H NMR (DMSO-d₆) δ ppm 13.96 (br s, 1H), 12.74 (br s, 1H), 8.02 (s, 1H), 7.68-7.65 (m, 1H), 7.55-7.49 (m, 3H); ¹³C NMR (DMSO-d₆) δ ppm 174.5, 152.8, 149.7, 141.4, 135.8, 131.9, 131.3, 130.72, 130.0, 128.3, 110.2; MS (ESI) m/z 279 (M+1).

### Example 12

### 3-(2-methoxyphenyl)-2-thioxo-1,2,3,7-tetrahydro-6H-purin-6-one

### (a) 6-Amino-1-(2-methoxyphenyl)-5-nitroso-2-thioxo-2,3-dihydropyrimidin-4(1H)-one

6-Amino-1-(2-methoxyphenyl)-2-thioxo-2,3-dihydropyrimidin-4(1H)-one (Ganaphthi et al., Proceedings A., Chemical Sciences, 1953, 37A, 652-9) (0.84 g, 3.4 mmol) was suspended in acetic acid (10% aq., 7 mL) and heated at 75° C. for 5 minutes. Sodium nitrite (0.33 g, 4.8 mmol), dissolved in water (2 mL), was added during 5 minutes and heating was continued for another 1 hour. Additional sodium nitrite (totally 0.30 g dissolved in 3 mL water) was added during 3 hours until the reaction was complete. The reaction mixture was diluted with water (10 mL) and the solid was collected by filtration, washed with water and dried to give the title compound (0.70 g, 75%) as a purple solid. The crude product was used in the next step without further purification.

¹H NMR (DMSO-d₆) δ ppm 12.96 (br s, 1H), 12.71 (br s, 1H), 8.05 (br s, 1H), 7.56-7.52 (m, 1H), 7.37-7.34 (m, 1H), 7.25 (d, 1H), 7.14-7.10 (m, 1H); 3.76 (s, 3H). MS (ESI) m/z 279 (M+1).

### (b) 5,6-Diamino-1-(2-methoxyphenyl)-2-thioxo-2,3-dihydropyrimidin-4(1H)-one

6-Amino-1-(2-methoxyphenyl)-5-nitroso-2-thioxo-2,3-dihydropyrimidin-4(1H)-one (0.70 g, 2.5 mmol, obtained from Example 12(a)) was suspended in water (5 mL) and ammonia (28% aq., 5 mL) was added. The reaction mixture was heated at 75° C. and sodium dithionite (1.1 g, 6.3 mmol) was added and stirring was continued at 75° C. for 15 minutes, and then stirred at r.t. for 0.5 hour. After adjusting the solution to neutral pH with 2M sulfuric acid, the precipitated solid was collected by filtration, washed with water and dried, giving the title compound (0.54 g, 80%). The crude product was used in the next step without further purification.

¹H NMR (DMSO-d₆) δ ppm 11.95 (br s, 1H), 7.49-7.45 (m, 1H), 7.21-7.18 (m, 2H), 7.09-7.05 (m, 1H), 5.37 (s, 2H), 3.76 (s, 3H), 3.43 (s, 2H); MS (ESI) m/z 265 (M+1).

### (c) 3-(2-methoxyphenyl)-2-thioxo-1,2,3,7-tetrahydro-6H-purin-6-one

A solution of 5,6-diamino-1-(3-methoxyphenyl)-2-thioxo-2,3-dihydropyrimidin-4(1H)-one (0.54 g, 2.0 mmol, obtained from Example 12(b)) and formamidine acetate (0.32 g, 3.1 mmol) in DMSO (3 mL) was heated to 70° C. for 2.5 h. The crude product mixture was purified by preparative HPLC, and then recrystallized from EtOH, giving the title compound (0.19 g, 35%) as a solid.

¹H NMR (DMSO-d₆) δ ppm 13.81 (br s, 1H), 12.58 (br s, 1H), 7.97 (s, 1H), 7.48-7.43 (m, 1H), 7.28-7.26 (m, 1H), 7.20-7.19 (m, 1H), 7.08-7.04 (m, 1H), 3.70 (s, 3H);

¹³C NMR (DMSO-d₆) δ ppm 175.0, 154.7, 152.9, 150.3, 141.2, 130.4, 130.1, 126.9, 120.6, 112.7, 110.2, 55.73; MS (ESI) m/z 275 (M+1).

### Example 13

### 2-thioxo-3-[6-(trifluoromethyl)pyridin-3-yl]-1,2,3,7-tetrahydro-6H-purin-6-one

### (a) 6-Amino-2-thioxo-1-[6-(trifluoromethyl)pyridin-3-yl]-2,3-dihydropyrimidin-4(1H)-one

To a solution of N-[6-(trifluoromethyl)pyridin-3-yl]thiourea (Love et al. PCT Int. Appl. (2001), WO2001064674) (0.94 g, 4.3 mmol) in ethanol (10 mL) was added, dropwise in portions during 9 h, sodium ethoxide (21% w/w, 8.0 mL, 21.4 mmol) and a solution of ethyl cyanoacetate (2.3 mL, 21.4 mmol) in ethanol (8 mL) while the reaction was stirred at 80° C. After cooling to r.t. water (100 mL) was added followed by 2M sulfuric acid to neutralize the reaction mixture, which afforded a precipitation as a sticky oil. The water phase was decanted of, dichloromethane (30 mL) was added and the formed solid was collected by filtration, giving the title compound (0.39 g, 31%). The crude product was used in the next step without further purification.

¹H NMR (DMSO-d₆) δ ppm 12.19 (s, 1H), 8.75 (d, 1H), 8.16-8.07 (m, 2H), 6.58 (br s, 2H), 5.01 (s, 1H); MS (ESI) m/z 289 (M+1).

### (b) 2-thioxo-3-[6-(trifluoromethyl)pyridin-3-yl]-1,2,3,7-tetrahydro-6H-purin-6-one

A suspension of 6-amino-2-thioxo-1-[6-(trifluoromethyl)pyridin-3-yl]-2,3-dihydropyrimidin-4(1H)-one (0.33 g, 1.1 mmol, obtained from Example 13(a)) in acetic acid (3 mL) and water (0.5 mL) was heated to 75° C. Sodium nitrite (0.30 g, 1.7 mmol) in water (0.5 ml) was added dropwise and the reaction mixture was stirred at r.t. for 20 minutes. Sodium dithionite (0.30 g, 1.7 mmol) was added and after 0.5 h the solvents was evaporated in vacuo, followed by evaporation of added toluene. DMSO (3 mL) and formamidine acetate (0.18 g, 1.7 mmol) was added to the residue and the reaction mixture was heated at 75° C. for 1.5 h. The crude product was purified by preparative HPLC, giving the title compound (0.072 g, 20%) as a solid.

¹H NMR (DMSO-d₆) δ ppm 13.98 (br s, 1H), 12.86 (br s, 1H), 8.87 (d, 1H), 8.27 (dd, 1H), 8.16 (d, 1H), 8.06 (s, 1H); ¹³C NMR (DMSO-d₆) δ ppm 175.8, 153.5, 151.5, 150.6, 146.5 (q), 141.9, 140.1, 138.9, 122.2, 122.1 (q), 111.4; MS (ESI) m/z 312 (M−1).

### Example 14

### 3-pyridin-3-yl-2-thioxo-1,2,3,7-tetrahydro-6H-purin-6-one

### (a) 6-Amino-1-pyridin-3-yl-2-thioxo-2,3-dihydropyrimidin-4(1H)-one

To a suspension of N-pyridin-3-ylthiourea (1.2 g, 8.0 mmol) in ethanol (10 mL) was added, dropwise in portions during 8 h, sodium ethoxide (21% w/w, 18 mL, 48.0 mmol) and a solution of ethyl cyanoacetate (5.1 mL, 48.0 mmol) in ethanol (10 mL) while the reaction was stirred at 80° C. After cooling to r.t. ethanol was removed in vacuo. Water (100 mL) was added and the solution was neutralized with 2M sulfinic acid. The formed solid was collected by filtration and washed with water giving the title compound (1.2 g, 70%). The crude product was used in the next step without further purification.

¹H NMR (DMSO-d₆) δ ppm 12.07 (s, 1H), 8.66 (dd, 1H), 8.50 (d, 1H), 7.80 (dd, 1H), 7.56 (dd, 1H), 6.44 (s, 2H), 5.00 (s, 1H); MS (ESI) m/z 221 (M+1).

### (b) 6-Amino-5-nitroso-1-pyridin-3-yl-2-thioxo-2,3-dihydropyrimidin-4(1H)-one

A suspension of 6-amino-1-pyridin-3-yl-2-thioxo-2,3-dihydropyrimidin-4(1H)-one (0.66 g, 3.0 mmol obtained from Example 14(a)) in acetic acid (5 mL) was heated to 80° C. Sodium nitrite (0.23 g, 3.3 mmol) in water (1 mL) was added dropwise and the reaction mixture was stirred at r.t. for 1.5 h. Additional sodium nitrite (0.10 g, 1.5 mmol) in water (0.5 mL) was added dropwise and stirring was continued for 20 minutes. Acetic acid (5 mL) was then added and solids were removed by filtration. Addition of water (50 mL) to the filtrate gave a solid, which was collected by filtration, giving the title compound (0.16 g, 21%). The crude product was used in the next step without further purification.

¹H NMR (DMSO-d₆) δ ppm 13.02 (s, 1H), 12.93 (s, 1H), 8.73 (d, 1H), 8.61 (s, 1H), 8.28 (s, 1H), 7.89 (d, 1H), 7.63 (dd, 1H); MS (ESI) m/z 250 (M+1).

### (c) 3-pyridin-3-yl-2-thioxo-1, z 3,7-tetrahydro-6H-purin-6-one

To a suspension of 6-amino-5-nitroso-1-pyridin-3-yl-2-thioxo-2,3-dihydropyrimidin-4(1H)-one (0.16 g, 0.64 mmol, obtained from Example 14(b)) in water (1 mL) was added ammonium hydroxide (concentrated, 2 mL). The reaction mixture was heated to 75° C. and sodium dithionite (0.22 g, 1.28 mmol) was added in small portions and then heating was continued for another 0.5 h. After cooling to r.t. the solvent was evaporated in vacuo, followed by the evaporation of added toluene. To the residue were DMSO (2 mL) and formamidine acetate (0.13 g, 1.28 mmol) added and the reaction mixture was heated to 75° C. for 1.5 h. The crude product was purified by preparative HPLC, giving the title compound (0.031 g, 20%) as a solid.

¹H NMR (DMSO-d₆) δ ppm 12.09 (br s, 2H), 8.58 (dd, 1H), 8.52 (d, 1H), 7.89 (s, 1H), 7.82 (dd, 1H), 7.52 (dd, 1H); ¹³C NMR (DMSO-d₆) δ ppm 175.4, 153.5, 150.7, 150.1, 149.7, 141.9, 137.3, 136.0, 124.4, 111.7; MS (ESI) m/z 246 (M+1).

### Screens

Methods for the determination of MPO inhibitory activity are disclosed in patent application WO 02/090575. The pharmacological activity of compounds according to the invention was tested in the following screen in which the compounds were either tested alone or in the presence of added tyrosine:

Assay buffer: 20 mM sodium/potassium phosphate buffer pH 6.5 containing 10 mM taurine and 100 mM NaCl.

Developing reagent: 2 mM 3,3′,5,5′-tetramethylbenzidine (TMB), 200 μM KI, 200 mM acetate buffer pH 5.4 with 20% DMF.

To 10 μl of diluted compounds in assay buffer, 40 μl of human MPO (final concentration 2.5 nM), with or without 20 μM tyrosine (final concentration, if present, 8 μM), was added and the mixture was incubated for 10 minutes at ambient temperature. Then 50 μl of H₂O₂ (final concentration 100 μM), or assay buffer alone as a control, were added. After incubation for 10 minutes at ambient temperature, the reaction was stopped by adding 10 μl 0.2 mg/ml of catalase (final concentration 18 μg/ml). The reaction mixture was left for an additional 5 minutes before 100 μl of TMB developing reagent was added. The amount of oxidised 3,3′,5,5′-tetramethylbenzidine formed was then measured after about 5 minutes using absorbance spectroscopy at about 650 nM. IC₅₀ values were then determined using standard procedures.

When tested in at least one version of the above screen, the compounds of Examples 1 to 14 gave IC₅₀ values of less than 60 μM, indicating that they are expected to show useful therapeutic activity. Representative results are shown in the following Table.

| | Inhibition of MPO (in the presence |
|---|---|
| Compound | of tyrosine) IC₅₀ μM |
| Example 3 | 2.75 |
| Example 8 | 4.47 |

1. A compound of Formula (I), or a pharmaceutically acceptable salt, solvate or solvate of a salt thereof 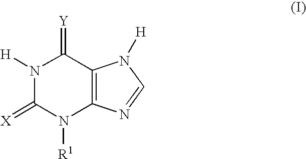
wherein
at least one of X and Y is S, and the other is O or S;
R¹ is an aromatic ring system selected from phenyl, biphenyl, naphthyl or 5 or 6 membered heteroaromatic ring containing one or more heteroatoms selected from N, O or S and said 5 or 6 membered heteroaromatic ring may optionally be fused with a 5 or 6 membered saturated, partially saturated or unsaturated ring containing one or more atoms selected from C, N, O or S, and wherein said 5 or 6 membered heteroaromatic ring alone or said 5 or 6 membered heteroaromatic ring fused with a 5 or 6 membered saturated, partially saturated or unsaturated ring is optionally substituted by one or more substituents independently selected from halogen, CHF₂, CH₂F, CF₃, SO₍ₙ₎R², SO₍ₙ₎NR²R³, S(O)ₙ, OH, OCF₃, C1 to 6 alkyl C1 to 6 alkoxy, CN, CONR⁴R⁵, NR⁴COR⁵ and COR⁵; said alkoxy being optionally further substituted by C1 to 6 alkoxy and said alkoxy optionally incorporating a carbonyl adjacent to the oxygen, and said alkyl being optionally further substituted by hydroxy or C1 to 6 alkoxy and said alkyl or alkoxy optionally incorporating a carbonyl adjacent to the oxygen or at any position in the alkyl;
with the proviso that when R¹ is phenyl, said phenyl must be substituted with one or more substituents independently selected from halogen, CHF₂, CH₂F, CF₃, SO₍ₙ₎R² SO₍ₙ₎NR²R³, OH, OCF₃, C1 to 6 alkyl C1 to 6 alkoxy, CN, CONR⁴R⁵, NR⁴COR⁵ and COR⁵; said alkoxy being optionally further substituted by C1 to 6 alkoxy and said alkoxy optionally incorporating a carbonyl adjacent to the oxygen, and said alkyl being optionally further substituted by hydroxy or C1 to 6 alkoxy and said alkyl or alkoxy optionally incorporating a carbonyl adjacent to the oxygen or at any position in the alkyl;
at each occurrence, R², R³, R⁴ and R⁵ independently represent hydrogen, C1 to 6 alkyl or C1 to 6 alkoxy said alkoxy optionally incorporating a carbonyl adjacent to the oxygen, said alkyl being optionally further substituted by halogen, C1 to 6 alkoxy, CHO, C2 to 6 alkanoyl, OH, CONR⁶R⁷ and NR⁶COR⁷;
or the groups NR⁴R⁵ and NR²R³ each independently represent a 5 to 7 membered saturated azacyclic ring optionally incorporating one additional heteroatom selected from O, S and NR⁸, said ring being optionally further substituted by halogen, C1 to 6 alkoxy, CHO, C2 to 6 alkanoyl, OH, CONR⁶R⁷ and NR⁶COR⁷;
at each occurrence R⁶, R⁷ and R⁸ independently represent hydrogen or C1 to 6 alkyl, or the group NR⁶R⁷ represents a 5 to 7 membered saturated azacyclic ring optionally incorporating one additional heteroatom selected from O, S and NR⁸; and
n is an integer 0, 1 or 2.

2. A compound according to claim 1, wherein X is S and Y is O.

3. A compound according to claim 1, wherein R¹ is a phenyl, substituted with one or more substituents independently selected from halogen, CHF₂, CH₂F, CF₃, SO₍ₙ₎R², SO₍ₙ₎NR²R³, OH, OCF₃, C1 to 6 alkyl, C1 to 6 alkoxy, CN, CONR⁴R⁵, NR⁴COR⁵ and COR⁵.

4. A compound according to claim 3, wherein R¹ is a phenyl substituted with one or two substituents selected from OCF₃, CN, halogen, methoxy and C1 to 6 alkyl.

5. A compound according to claim 1, wherein R¹ is pyridyl optionally substituted by one or more substituents independently selected from halogen, CF₃, C1 to 6 alkyl and C1 to 6 alkoxy.

6. A compound, said compound being:
3-(3-chlorophenyl)-2-thioxo-1,2,3,7-tetrahydro-6H-purin-6-one;
3-(3-ethylphenyl)-2-thioxo-1,2,3,7-tetrahydro-6H-purin-6-one;
2-thioxo-3-[3-(trifluoromethoxy)phenyl]-1,2,3,7-tetrahydro-6H-purin-6-one;
3-(4-chlorophenyl)-2-thioxo-1,2,3,7-tetrahydro-6H-purin-6-one;
3-(3,5-dichlorophenyl)-2-thioxo-1,2,3,7-tetrahydro-6H-purin-6-one;
3-(6-oxo-2-thioxo-1,2,6,7-tetrahydro-3H-purin-3-yl)benzonitrile;
3-(4-methoxyphenyl)-2-thioxo-1,2,3,7-tetrahydro-6H-purin-6-one;
3-quinolin-3-yl-2-thioxo-1,2,3,7-tetrahydro-6H-purin-6-one;
3-(2,5-difluorophenyl)-2-thioxo-1,2,3,7-tetrahydro-6H-purin-6-one;
3-(3-fluorophenyl)-2-thioxo-1,2,3,7-tetrahydro-6H-purin-6-one;
3-(2-chlorophenyl)-2-thioxo-1,2,3,7-tetrahydro-6H-purin-6-one;
3-(2-methoxyphenyl)-2-thioxo-1,2,3,7-tetrahydro-6H-purin-6-one;
2-thioxo-3-[6-(trifluoromethyl)pyridin-3-yl]-1,2,3,7-tetrahydro-6H-purin-6-one, and
3-pyridin-3-yl-2-thioxo-1,2,3,7-tetrahydro-6H-purin-6-one;
or a pharmaceutically acceptable salt, solvate or solvate of a salt thereof.

7. A pharmaceutical composition comprising a compound according to claim 6, or a pharmaceutically acceptable salt thereof, optionally in admixture with a pharmaceutically acceptable adjuvant, diluent or carrier.

8. A pharmaceutical composition comprising a compound according to claim 1, or a pharmaceutically acceptable salt thereof, optionally in admixture with a pharmaceutically acceptable adjuvant, diluent or carrier.

9. A method of treating, or reducing the risk of, diseases or conditions in which inhibition of the enzyme MPO is beneficial which comprises administering to a person suffering from or at risk of, said disease or condition, a therapeutically effective amount of a compound as defined in claim 1, or a pharmaceutically acceptable salt thereof.

10. A method of treating, or reducing the risk of neuroinflammatory disorders which comprises administering to a person suffering from or at risk of, said disease or condition, a therapeutically effective amount of a compound as defined in claim 1, or a pharmaceutically acceptable salt thereof.

11. The method according to claim 10, wherein said neuroinflammatory disorder is multiple sclerosis.

12. The method according to claim 10, wherein said neuroinflammatory disorder is Parkinson's disease.

13. A method of treating, or reducing the risk of cardio- and cerebrovascular atherosclerotic disorders or peripheral artery disease and heart failure and respiratory disorders which comprises administering to a person suffering from or at risk of, said disease or condition, a therapeutically effective amount of a compound as defined in claim 1, or a pharmaceutically acceptable salt thereof.

14. The method according to claim 13, wherein said disease or condition is atherosclerosis.

15. The method according to claim 13, wherein said disease or condition is chronic obstructive pulmonary disease (COPD).

16. The method according to claim 13, wherein said disease or condition is bronchitis; emphysema; bronchiectasis; and/or cystic fibrosis.

17-24. (canceled)
